(19) United States
(12) Patent Application Publication
(10) Pub. No.: US 2005/0032750 A1
(43) Pub. Date: Feb. 10, 2005

(54) Treating androgen deficiency in female (ADIF)-associated conditions with SARMS

(76) Inventors: Mitchell S. Steiner, Germantown, TN (US); Karen A. Veverka, Cordova, TN (US); James T. Dalton, Upper Arlington, OH (US); Duane D. Miller, Germantown, TN (US)

Correspondence Address: EITAN, PEARL, LATZER & COHEN ZEDEK LLP, 10 ROCKEFELLER PLAZA, SUITE 1001, NEW YORK, NY, 10020, US

(21) Appl. No.: 10/759,538

(22) Filed: Jan. 20, 2004

Related U.S. Application Data
(60) Provisional application No. 60/441,308, filed on Jan. 22, 2003.

(51) Int. Cl. A61K 31/66; A61K 31/277; A61K 31/195; A61K 31/165

(52) U.S. Cl. 514/114; 514/522; 514/563; 514/616; 514/617; 514/602; 514/493

(57) ABSTRACT

The present invention provides a method of treating, preventing, suppressing, inhibiting or reducing the incidence of an Androgen Deficiency in Female (ADIF)-associated condition in a female subject, by administering to the subject a selective androgen receptor modulator (SARM) compound and/or its analog, derivative, isomer, metabolite, pharmaceutically acceptable salt, pharmaceutical product, hydrate, N-oxide, crystal, polymorph, prodrug, or any combination thereof. The present invention further provides a method of treating, preventing, suppressing, inhibiting or reducing the incidence of sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and ovarian cancer due to ADIF in a female subject, by administering to the subject a selective androgen receptor modulator (SARM) compound and/or its analog, derivative, isomer, metabolite, pharmaceutically acceptable salt, pharmaceutical product, hydrate, N-oxide, crystal, polymorph, prodrug, or any combination thereof.

### CROSS REFERENCE TO RELATED APPLICATIONS

This application claims the priority of United States Provisional Application Ser. No. 60/441,308, filed Jan. 22, 2003, which is incorporated in its entirety by reference herein.

### FIELD OF INVENTION

This invention generally relates to the prevention and treatment of Androgen Deficiency in Female (ADIF)-associated conditions in a female subject. More particularly, this invention relates to a method of treating, preventing, suppressing, inhibiting, or reducing an ADIF-associated condition in a female subject, for example sexual dysfunction, decreased sexual libido, hypogonadism, sarcopenia, osteoperiia, osteoporosis, alterations in cognition and mood, fatigue, depression, anemia, muscle weakness, hair loss, obesity, polycystic ovarian disease, endometriosis, breast cancer, uterine cancer and ovarian cancer, by administering to the female subject a selective androgen receptor modulator (SARM) compound and/or its analog, derivative, isomer, metabolite, pharmaceutically acceptable salt, pharmaceutical product, hydrate, N-oxide, crystal, polymorph, prodrug, or any combination thereof.

### BACKGROUND OF THE INVENTION

Androgen Deficiency in Female (ADIF) refers to a progressive decrease in androgen production, occurring in women after middle age, and/or in women undergoing chemotherapy and/or HIV-positive premenopausal women. In addition, various conditions can lead to ADIF such as Addison's disease, hypopituitary states, and ovariectomy (removal of ovaries). The syndrome is characterized by alterations in the physical, emotional and intellectual domains that correlate with and can be corrected by manipulation of the androgen milieu.

ADIF is characterized biochemically by a decrease not only in serum androgen, but also in other hormones, such as growth hormone, melatonin and dehydroepiandrosterone. Clinical manifestations include sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and ovarian cancer.

The onset of ADIF is unpredictable and its manifestations are subtle and variable, which has led to a paucity of interest in its diagnosis, monitoring and treatment.

Innovative approaches are urgently needed at both the basic science and clinical levels to treat ADIF. The present invention is directed to satisfying this need.

### SUMMARY OF THE INVENTION

The present invention provides a method of treating, preventing, suppressing, inhibiting or reducing the incidence of an Androgen Deficiency in Female (ADIF)-associated condition in a female subject, by administering to the subject a selective androgen receptor modulator (SARM) compound and/or its analog, derivative, isomer, metabolite, pharmaceutically acceptable salt, pharmaceutical product, hydrate, N-oxide, crystal, polymorph, prodrug, or any combination thereof. The present invention further provides a method of treating, preventing, suppressing, inhibiting or reducing the incidence of sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and ovarian cancer due to ADIF in a female subject, by administering to the subject a selective androgen receptor modulator (SARM) compound and/or its analog, derivative, isomer, metabolite, pharmaceutically acceptable salt, pharmaceutical product, hydrate, N-oxide, crystal, polymorph, prodrug, or any combination thereof.

In one embodiment, this invention relates to a method of treating a female subject suffering from an Androgen Deficiency in Female (ADIF)-associated condition, comprising the step of administering to the subject a selective androgen receptor modulator (SARM) compound, in an amount effective to treat the ADIF-associated condition. In another embodiment, the method comprises administering an analog, derivative, isomer, metabolite, pharmaceutically acceptable salt, hydrate, N-oxide, prodrug, polymorph, crystal, or any combination thereof of the SARM compound.

In another embodiment, the present invention provides a method of preventing, suppressing, inhibiting or reducing the incidence of an ADIF-associated condition in a female subject, comprising the step of administering to the subject a selective androgen receptor modulator (SARM) compound, in an amount effective to prevent, suppress, inhibit or reduce the incidence of the ADIF-associated condition. In another embodiment, the method comprises administering an analog, derivative, isomer, metabolite, pharmaceutically acceptable salt, hydrate, N-oxide, prodrug, polymorph, crystal, or any combination thereof of the SARM compound.

In another embodiment, the present invention provides a method of treating a female subject suffering from sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and/or ovarian cancer due to Androgen Deficiency in Female (ADIF), comprising the step of administering to the subject a selective androgen receptor modulator (SARM) compound. In another embodiment, the method comprises administering an analog, derivative, isomer, metabolite, pharmaceutically acceptable salt, hydrate, N-oxide, prodrug, polymorph, crystal, or any combination thereof of the SARM compound.

In another embodiment, the present invention provides a method of preventing, suppressing, inhibiting or reducing the incidence of an ADIF-associated condition selected from sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and/or ovarian cancer in a female subject, comprising the step of administering to the subject a selective androgen receptor modulator (SARM) compound.

In another embodiment, the method comprises administering an analog, derivative, isomer, metabolite, pharmaceutically acceptable salt, hydrate, N-oxide, prodrug, polymorph, crystal, or any combination thereof of the SARM compound.

In one embodiment, the SARM compound that is effective at treating, preventing, suppressing, inhibiting or reducing the incidence of the ADIF-associated condition is a compound represented by the structure of formula I:

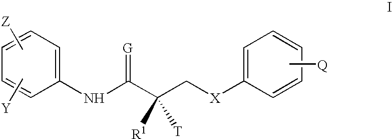

wherein G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃; F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 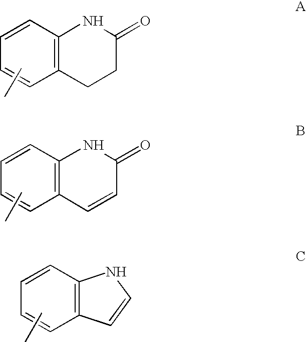
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CBF₂, CF₃, CH₂CH₃, or CF₂CF₃;

or its analog, derivative, isomer, metabolite, pharmaceutically acceptable salt, pharmaceutical product, hydrate, N-oxide, crystal, polymorph, prodrug, or any combination thereof.

In another embodiment, the SARM compound that is effective at treating, preventing, suppressing, inhibiting or reducing the incidence of the ADIF-associated condition is a compound represented by the structure of formula II:

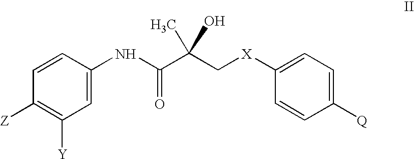

wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 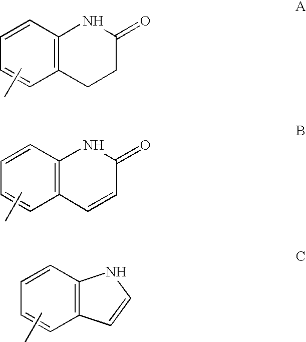
R is alkyl, haloalkyl, dihaloalkyl, trihaloalkyl, CH₂F, CHF₂, CF₃, CF₂CF₃, aryl, phenyl, halogen, alkenyl or OH;

or its analog, derivative, isomer, metabolite, pharmaceutically acceptable salt, pharmaceutical product, hydrate, N-oxide, crystal, polymorph, prodrug, or any combination thereof.

In another embodiment, the SARM compound that is effective at treating, preventing, suppressing, inhibiting or reducing the incidence of the ADIF-associated condition is a compound represented by the structure of formula III:

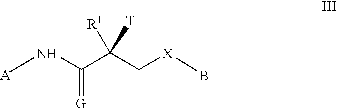

wherein X is a bond, O, CH₂, NH, Se, PR, NO or NR;
G is O or S;
R₁ is CH₃, CH₂F, CBF₂, CF₃, CH₂CH₃, or CF₂CF₃;
T is OH, OR, —NHCOCH₃, or NHCOR;
R is alkyl; haloalkyl, dihaloalkyl, trihaloalkyl, CH₂F, CHF₂, CF₃, CF₂CF₃, aryl, phenyl, halogen, alkenyl or OH;
A is a ring selected from: 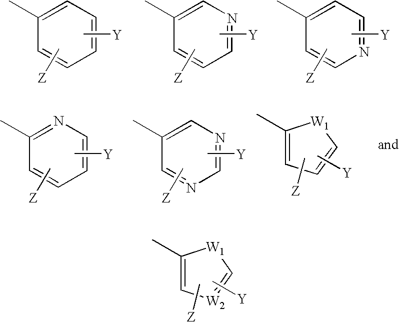
B is a ring selected from:

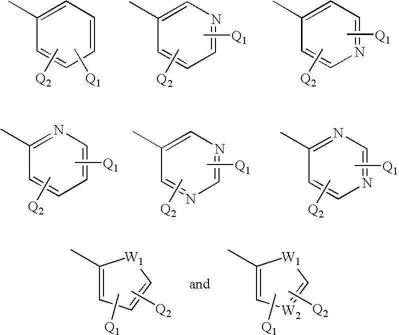

wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 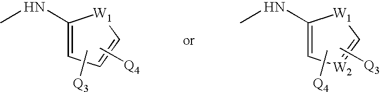
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO;

or its analog, derivative, isomer, metabolite, pharmaceutically acceptable salt, pharmaceutical product, hydrate, N-oxide, crystal, polymorph, prodrug, or any combination thereof.

In another embodiment, the SARM compound that is effective at treating, preventing, suppressing, inhibiting or reducing the incidence of the ADIF-associated condition is a compound represented by the structure of formula IV:

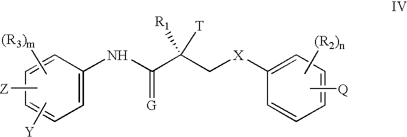

wherein X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 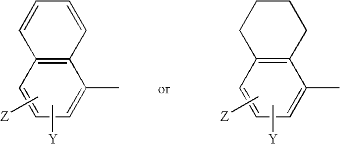
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 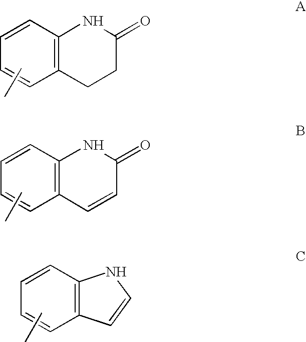
n is an integer of 1-4; and
m is an integer of 1-3;

or its analog, derivative, isomer, metabolite, pharmaceutically acceptable salt, pharmaceutical product, hydrate, N-oxide, crystal, polymorph, prodrug, or any combination thereof.

In another embodiment, the SARM compound that is effective at treating, preventing, suppressing, inhibiting or reducing the incidence of the ADIF-associated condition is a compound represented by the structure of formula V: 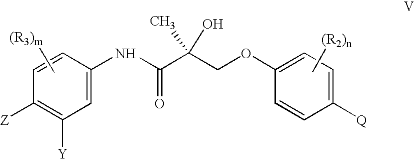
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 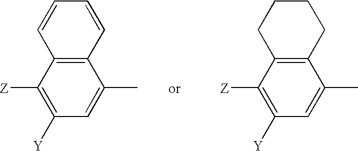
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 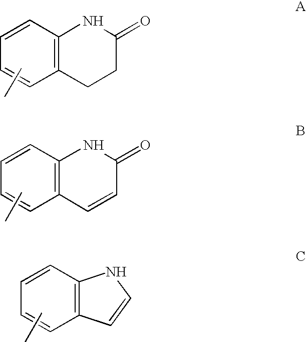
n is an integer of 1-4; and
m is an integer of 1-3;

or its analog, derivative, isomer, metabolite, pharmaceutically acceptable salt, pharmaceutical product, hydrate, N-oxide, crystal, polymorph, prodrug, or any combination thereof.

In another embodiment, the SARM compound that is effective at treating, preventing, suppressing, inhibiting or reducing the incidence of the ADIF-associated condition is a compound represented by the structure of formula VI, or its analog, derivative, isomer, metabolite, pharmaceutically acceptable salt, pharmaceutical product, hydrate, N-oxide, crystal, polymorph, prodrug, or any combination thereof.

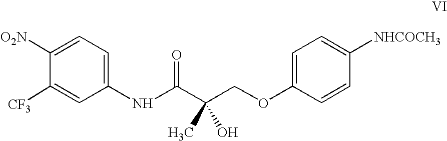

In another embodiment, the SARM compound that is effective at treating, preventing, suppressing, inhibiting or reducing the incidence of the ADIF-associated condition is a compound represented by the structure of formula VII, or its analog, derivative, isomer, metabolite, pharmaceutically acceptable salt, pharmaceutical product, hydrate, N-oxide, crystal, polymorph, prodrug, or any combination thereof.

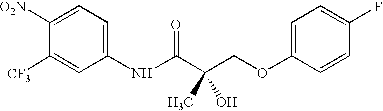

### VII

In another embodiment, the SARM compound that is effective at treating, preventing, suppressing, inhibiting or reducing the incidence of the ADIF-associated condition is a compound represented by the structure of formula VIII, or its analog, derivative, isomer, metabolite, pharmaceutically acceptable salt, pharmaceutical product, hydrate, N-oxide, crystal, polymorph, prodrug, or any combination thereof.

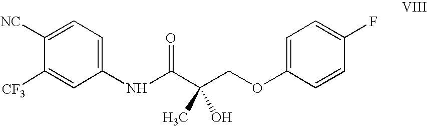

In another embodiment, the SARM compound that is effective at treating, preventing, suppressing, inhibiting or reducing the incidence of the ADIF-associated condition is a compound represented by the structure of formula IX, or its analog, derivative, isomer, metabolite, pharmaceutically acceptable salt, pharmaceutical product, hydrate, N-oxide, crystal, polymorph, prodrug, or any combination thereof.

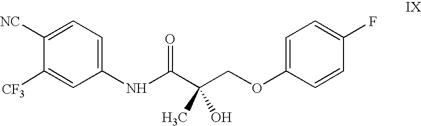

In another embodiment, the SARM compound that is effective at treating, preventing, suppressing, inhibiting or reducing the incidence of the ADIF-associated condition is a compound represented by the structure of formula X, or its analog, derivative, isomer, metabolite, pharmaceutically acceptable salt, pharmaceutical product, hydrate, N-oxide, crystal, polymorph, prodrug, or any combination thereof.

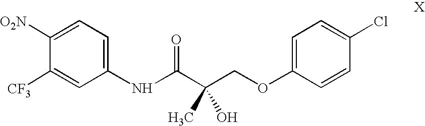

In another embodiment, the SARM compound that is effective at treating, preventing, suppressing, inhibiting or reducing the incidence of the ADIF-associated condition is a compound represented by the structure of formula A, or its analog, derivative, isomer, metabolite, pharmaceutically acceptable salt, pharmaceutical product, hydrate, N-oxide, crystal, polymorph, prodrug, or any combination thereof.

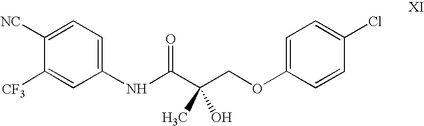

In one embodiment, the SARM is an androgen receptor agonist. In another embodiment, the SARM has in-vivo androgenic and anabolic activity of a nonsteroidal ligand for the androgen receptor. In another embodiment, the SARM is an androgen receptor antagonist. In another embodiment, the SARM has antagonistic effect muscle or bone. In another embodiment, the SARM has no effect on muscle or bone. In another embodiment, the SARM penetrates the central nervous system (CNS). In another embodiment, the SARM does not penetrate the central nervous system (CNS).

In one embodiment, the ADIF-associated condition is sexual dysfunction. In another embodiment, the ADIF-associated condition is decreased sexual libido. In another embodiment, the ADIF-associated condition is hypogonadism. In another embodiment, the ADIF-associated condition is sarcopenia. In another embodiment, the ADIF-associated condition is osteopenia. In another embodiment, the ADIF-associated condition is osteoporosis. In another embodiment, the ADIF-associated condition comprises alterations in cognition and mood. In another embodiment, the ADIF-associated condition is fatigue. In another embodiment, the ADIF-associated condition is depression. In another embodiment, the ADIF-associated condition is anemia. In another embodiment, the ADIF-associated condition is hair loss. In another embodiment, the ADIF-associated condition is muscle weakness. In another embodiment, the ADIF-associated condition is obesity. In another embodiment, the ADIF-associated condition is polycystic ovarian disease. In another embodiment, the ADIF-associated condition is endometriosis. In another embodiment, the ADIF-associated condition is breast cancer. In another embodiment, the ADIF-associated condition is uterine cancer. In another embodiment, the ADIF-associated condition is ovarian cancer. In another embodiment, the ADIF-associated condition is any combination of the conditions recited hereinabove.

The present invention provides a safe and effective method of treating, preventing, suppressing, inhibiting or reducing the incidence of ADIF-associated conditions and is particularly useful in treating female subjects suffering from symptoms and signs of sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and ovarian cancer.

### BRIEF DESCRIPTION OF THE DRAWINGS

The present invention will be understood and appreciated more fully from the 2 5 following detailed description taken in conjunction with the appended drawings in which:

FIG. 1: Effect of testosterone proprionate and Compound VI on mysoin heavy chain (MHC) IIb mRNA expression. FIG. 1A: histogram showing effect of Compound VI on MHC IIb MRNA expression; and FIG. 1B: RT-PCR showing m-RNA expression of MHC IIb.

FIG. 2: Effect of SARMS on Bone Mineral Content (BMC) and Bone Mineral Density (BMD) in female rates after ovariectomy.

FIG. 3: Effect of Compound VI on total body Bone Mineral Density (BMD) in female rats.

FIG. 4: Effect of Compound VI on body weight.

FIG. 5: Percent fat mass at day 120 following treatment with Compound VI.

FIG. 6: Bone sparing effect of Compound VI in the vertebrae L2-L4.

FIG. 7: Compound VI prevents the loss of Cortical Content (CC) of the mid-shaft femur following ovariectomy.

FIG. 8: Femoral 3pt bending—effect of compound VI on femur strength.

### DETAILED DESCRIPTION OF THE INVENTION

The present invention provides a method of treating, preventing, suppressing, inhibiting or reducing the incidence of an Androgen Deficiency in Female (ADIF)-associated condition in a female subject, by administering to the subject a selective androgen receptor modulator (SARM) compound and/or its analog, derivative, isomer, metabolite, pharmaceutically acceptable salt, pharmaceutical product, hydrate, N-oxide, crystal, polymorph, prodrug, or any combination thereof. The present invention further provides a method of treating, preventing, suppressing, inhibiting or reducing the incidence of sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and ovarian cancer due to ADIF in a female subject, by administering to the subject a selective androgen receptor modulator (SARM) compound and/or its analog, derivative, isomer, metabolite, pharmaceutically acceptable salt, pharmaceutical product, hydrate, N-oxide, crystal, polymorph, prodrug, or any combination thereof

Thus, in one embodiment, this invention relates to a method of treating a female subject suffering from an Androgen Deficiency in Female (ADIF)-associated condition, comprising the step of administering to the subject a selective androgen receptor modulator (SARM) compound, in an amount effective to treat the ADIF-associated condition. In another embodiment, the method comprises administering an analog, derivative, isomer, metabolite, pharmaceutically acceptable salt, hydrate, N-oxide, prodrug, polymorph, crystal, or any combination thereof of the SARM compound.

In another embodiment, the present invention provides a method of preventing, suppressing, inhibiting or reducing the incidence of an ADIF-associated condition in a female subject, comprising the step of administering to the subject a selective androgen receptor modulator (SARM) compound, in an amount effective to prevent, suppress, inhibit or reduce the incidence of the ADIF-associated condition. In another embodiment, the method comprises administering an analog, derivative, isomer, metabolite, pharmaceutically acceptable salt, hydrate, N-oxide, prodrug, polymorph, crystal, or any combination thereof of the SARM compound.

In another embodiment, the present invention provides a method of treating a female subject suffering from sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and/or ovarian cancer due to Androgen Deficiency in Female (ADIF), comprising the step of administering to the subject a selective androgen receptor modulator (SARM) compound. In another embodiment, the method comprises administering an analog, derivative, isomer, metabolite, pharmaceutically acceptable salt, hydrate, N-oxide, prodrug, polymorph, crystal, or any combination thereof of the SARM compound.

In another embodiment, the present invention provides a method of preventing, suppressing, inhibiting or reducing the incidence of an ADIF-associated condition selected from sexual dysfimction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and/or ovarian cancer in a female subject, comprising the step of administering to the subject a selective androgen receptor modulator (SARM) compound. In another embodiment, the method comprises administering an analog, derivative, isomer, metabolite, pharmaceutically acceptable salt, hydrate, N-oxide, prodrug, polymorph, crystal, or any combination thereof of the SARM compound.

In one embodiment, the SARM compound that is effective at treating, preventing, suppressing, inhibiting or reducing the incidence of the ADIF-associated condition is a compound represented by the structure of formula I:

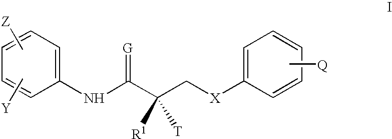

wherein G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 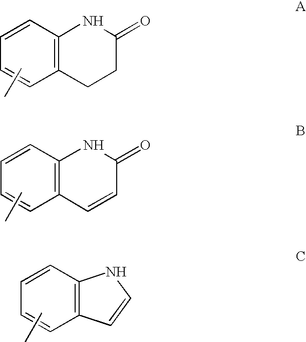
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

In one embodiment, the SARM is an analog of the compound of formula I. In another embodiment, the SARM is a derivative of the compound of formula I. In another embodiment, the SARM is an isomer of the compound of formula I. In another embodiment, the SARM is a metabolite of the compound of formula I. In another embodiment, the SARM is a pharmaceutically acceptable salt of the compound of formula I. In another embodiment, the SARM is a pharmaceutical product of the compound of formula I. In another embodiment, the SARM is a hydrate of the compound of formula I. In another embodiment, the SARM is an N-oxide of the compound of formula I. In another embodiment, the SARM is a crystal of the compound of formula I. In another embodiment, the SARM is a polymorph of the compound of formula I. In another embodiment, the SARM is a prodrug of the compound of formula I. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula I.

In one embodiment, the SARM compound is a compound of formula I wherein X is O. In one embodiment, the SARM compound is a compound of formula I wherein G is O. In another embodiment, the SARM compound is a compound of formula I wherein Z is NO₂. In another embodiment, the SARM compound is a compound of formula I wherein Z is CN. In another embodiment, the SARM compound is a compound of formula I wherein Y is CF₃. In another embodiment, the SARM compound is a compound of formula I wherein Q is NHCOCH₃. In another embodiment, the SARM compound is a compound of formula I wherein Q is F. In another embodiment, the SARM compound is a compound of formula I wherein T is OH. In another embodiment, the SARM compound is a compound of formula I wherein R₁ is CH₃.

The substituents Z and Y can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituent Q can be in any position of the ring carrying this substituent (hereinafter “B ring”). In one embodiment, the substituent Q is in the para position of the B ring. In another embodiment, the substituent Q is NHCOCH₃ and is in the para position of the B ring. In another embodiment, the substituent Q is F and is in the para position of the B ring.

In another embodiment, the SARM compound that is effective at treating, preventing, suppressing, inhibiting or reducing the incidence of the ADIF-associated condition is a compound represented by the structure of formula II:

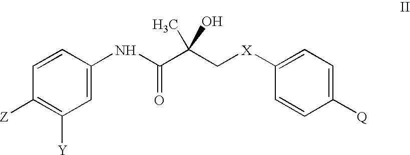

wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 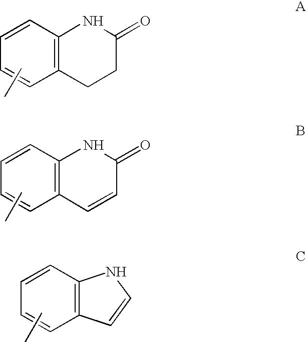
R is alkyl, haloalkyl, dihaloalkyl, trihaloalkyl, CH₂F, CHF₂, CF₃, CF₂CF₃, aryl, phenyl, halogen, alkenyl or OH.

In one embodiment, the SARM is an analog of the compound of formula II. In another embodiment, the SARM is a derivative of the compound of formula II. In another embodiment, the SARM is an isomer of the compound of formula II. In another embodiment, the SARM is a metabolite of the compound of formula II. In another embodiment, the SARM is a pharmaceutically acceptable salt of the compound of formula II. In another embodiment, the SARM is a pharmaceutical product of the compound of formula II. In another embodiment, the SARM is a hydrate of the compound of formula II. In another embodiment, the SARM is an N-oxide of the compound of formula II. In another embodiment, the SARM is a crystal of the compound of formula II. In another embodiment, the SARM is a polymorph of the compound of formula II. In another embodiment, the SARM is a prodrug of the compound of formula II. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula II.

In one embodiment, the SARM compound is a compound of formula II wherein X is O. In another embodiment, the SARM compound is a compound of formula II wherein Z is NO₂. In another embodiment, the SARM compound is a compound of formula II wherein Z is CN. In another embodiment, the SARM compound is a compound of formula II wherein Y is CF₃. In another embodiment, the SARM compound is a compound of formula II wherein Q is NHCOCH₃. In another embodiment, the SARM compound is a compound of formula II wherein Q is F.

In another embodiment, the SARM compound that is effective at treating, preventing, suppressing, inhibiting or reducing the incidence of the ADIF-associated condition is a compound represented by the structure of formula III:

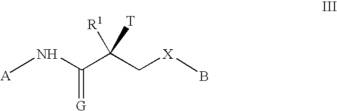

wherein X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 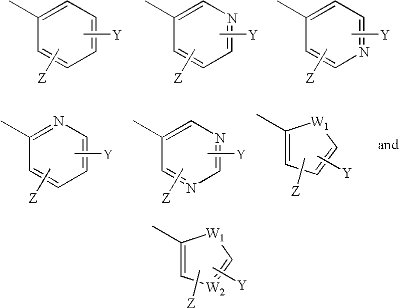
B is a ring selected from:

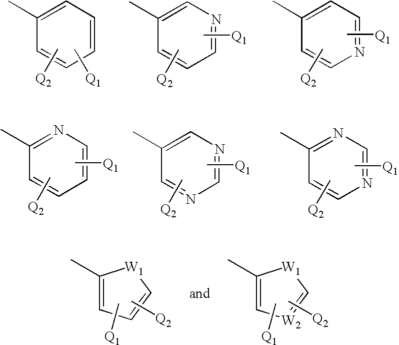

wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 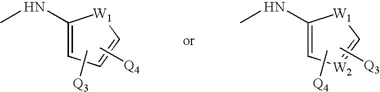
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

In one embodiment, the SARM is an analog of the compound of formula III. In another embodiment, the SARM is a derivative of the compound of formula III. In another embodiment, the SARM is an isomer of the compound of formula III. In another embodiment, the SARM is a metabolite of the compound of formula III. In another embodiment, the SARM is a pharmaceutically acceptable salt of the compound of formula III. In another embodiment, the SARM is a pharmaceutical product of the compound of formula III. In another embodiment, the SARM is a hydrate of the compound of formula III. In another embodiment, the SARM is an N-oxide of the compound of formula III. In another embodiment, the SARM is a crystal of the compound of formula III. In another embodiment, the SARM is a polymorph of the compound of formula III. In another embodiment, the SARM is a prodrug of the compound of formula III. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula III.

In one embodiment, the SARM compound is a compound of formula III wherein X is O. In another embodiment, the SARM compound is a compound of formula III wherein G is O. In another embodiment, the SARM compound is a compound of formula I wherein T is OH. In another embodiment, the SARM compound is a compound of formula III wherein R₁ is CH₃. In another embodiment, the SARM compound is a compound of formula III wherein Z is NO₂. In another embodiment, the SARM compound is a compound of formula III wherein Z is CN. In another embodiment, the SARM compound is a compound of formula III wherein Y is CF₃. In another embodiment, the SARM compound is a compound of formula HI wherein Q₁ is NHCOCH₃. In another embodiment, the SARM compound is a compound of formula III wherein Q₁ is F.

The substituents Z and Y can be in any position of the ring carrying these substituents (hereinafter “A ring”). In one embodiment, the substituent Z is in the pars position of the A ring. In another embodiment, the substituent Y is in the meta position of the A ring. In another embodiment, the substituent Z is in the para position of the A ring and substituent Y is in the meta position of the A ring.

The substituents Q₁ and Q₂ can be in any position of the ring carrying these substituents (hereinafter “B ring”). In one embodiment, the substituent Q₁ is in the para position of the B ring. In another embodiment, the substituent is Q₂ is H. In another embodiment, the substituent Q₁ is in the para position of the B ring and the substituent is Q₂ is H. In another embodiment, the substituent Q₁ is NHCOCH₃ and is in the para position of the B ring, and the substituent is Q₂ is H.

In another embodiment, the SARM compound that is effective at treating, preventing, suppressing, inhibiting or reducing the incidence of the ADIF-associated condition is a compound represented by the structure of formula IV:

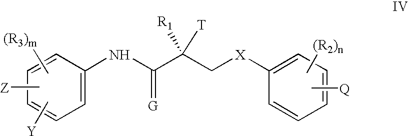

wherein X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 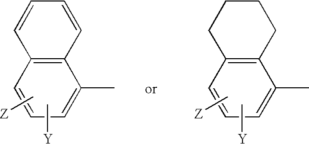
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 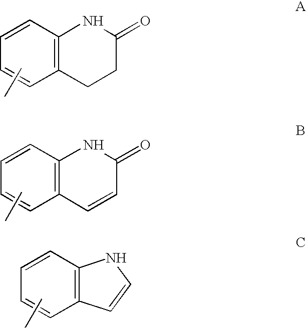
n is an integer of 1-4; and
m is an integer of 1-3.

In one embodiment, the SARM is an analog of the compound of formula IV. In another embodiment, the SARM is a derivative of the compound of formula IV. In another embodiment, the SARM is an isomer of the compound of formula IV. In another embodiment, the SARM is a metabolite of the compound of formula IV. In another embodiment, the SARM is a pharmaceutically acceptable salt of the compound of formula IV. In another embodiment, the SARM is a pharmaceutical product of the compound of formula IV. In another embodiment, the SARM is a hydrate of the compound of formula IV. In another embodiment, the SARM is an N-oxide of the compound of formula IV. In another embodiment, the SARM is a crystal of the compound of formula IV. In another embodiment, the SARM is a polymorph of the compound of formula IV. In another embodiment, the SARM is a prodrug of the compound of formula IV. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula IV.

In one embodiment, the SARM compound is a compound of formula IV wherein X is O. In another embodiment, the SARM compound is a compound of formula IV wherein G is O. In another embodiment, the SARM compound is a compound of formula IV wherein Z is NO₂. In another embodiment, the SARM compound is a compound of formula IV wherein Z is CN. In another embodiment, the SARM compound is a compound of formula IV wherein Y is CF₃. In another embodiment, the SARM compound is a compound of formula IV wherein Q is NHCOCH₃. In another embodiment, the SARM compound is a compound of formula IV wherein Q is F. In another embodiment, the SARM compound is a compound of formula IV wherein T is OH. In another embodiment, the SARM compound is a compound of formula IV wherein R₁ is CH₃. In another embodiment, the SARM compound is a compound of formula IV wherein Q is F and R₂ is CH₃. In another embodiment, the SARM compound is a compound of formula IV wherein Q is F and R₂ is Cl.

The substituents Z, Y and R₃ can be in any position of the ring carrying these substituents (hereinafter “A ring”). In one embodiment, the substituent Z is in the para position of the A ring. In another embodiment, the substituent Y is in the meta position of the A ring. In another embodiment, the substituent Z is in the para position of the A ring and substituent Y is in the meta position of the A ring.

The substituents Q and R₂ can be in any position of the ring carrying these substituents (hereinafter “B ring”). In one embodiment, the substituent Q is in the para position of the B ring. In another embodiment, the substituent Q is in the para position of the B ring. In another embodiment, the substituent Q is NHCOCH₃ and is in the para position of the B ring.

As contemplated herein, when the integers m and n are greater than one, the substituents R₂ and R₃ are not limited to one particular substituent, and can be any combination of the substituents listed above.

In another embodiment, the SARM compound that is effective at treating, preventing, suppressing, inhibiting or reducing the incidence of the ADIF-associated condition is a compound represented by the structure of formula V:

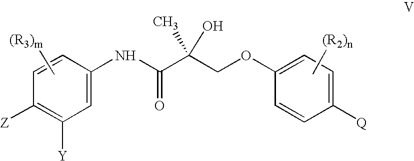

wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 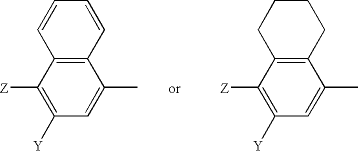
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃ F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 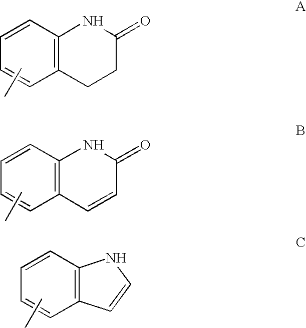
n is an integer of 1-4; and
m is an integer of 1-3.

In one embodiment, the SARM is an analog of the compound of formula V. In another embodiment, the SARM is a derivative of the compound of formula V. In another embodiment, the SARM is an isomer of the compound of formula V. In another embodiment, the SARM is a metabolite of the compound of formula V. In another embodiment, the SARM is a pharmaceutically acceptable salt of the compound of formula V. In another embodiment, the SARM is a pharmaceutical product of the compound of formula V. In another embodiment, the SARM is a hydrate of the compound of formula V. In another embodiment, the SARM is an N-oxide of the compound of formula V. In another embodiment, the SARM is a crystal of the compound of formula V. In another embodiment, the SARM is a polymorph of the compound of formula V. In another embodiment, the SARM is a prodrug of the compound of formula V. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula V.

In another embodiment, the SARM is a compound of formula V wherein Z is NO₂. In another embodiment, the SARM is a compound of formula V wherein Z is CN. In another embodiment, the SARM is a compound of formula V wherein Y is CF₃. In another embodiment, the SARM is a compound of formula V wherein Q is NHCOCH₃. In another embodiment, the SARM is a compound of formula V wherein Q is F. In another embodiment, the SARM is a compound of formula V wherein Q is F and R₂ is CH₃. In another embodiment, the SARM is a compound of formula V wherein Q is F and R₂ is Cl.

The substituents Z, Y and R₃ can be in any position of the A ring, and the substituents Q and R₂ can be in any position of B ring, as discussed above for compound IV. Furthermore, as discussed above, when the integers m and n are greater than one, the substituents R₂ and R₃ are not limited to one particular substituent, and can be any combination of the substituents listed above.

In another embodiment, the SARM compound that is effective at treating, preventing, suppressing, inhibiting or reducing the incidence of the ADIF-associated condition is a compound represented by the structure of formula VI.

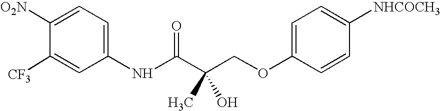

### VI

In one embodiment, the SARM is an analog of the compound of formula VI. In another embodiment, the SARM is a derivative of the compound of formula VI. In another embodiment, the SARM is an isomer of the compound of formula VI. In another embodiment, the SARM is a metabolite of the compound of formula VI. In another embodiment, the SARM is a pharmaceutically acceptable salt of the compound of formula VI. In another embodiment, the SARM is a pharmaceutical product of the compound of formula VI. In another embodiment, the SARM is a hydrate of the compound of formula VI. In another embodiment, the SARM is an N-oxide of the compound of formula VI. In another embodiment, the SARM is a crystal of the compound of formula VI. In another embodiment, the SARM is a polymorph of the compound of formula VI. In another embodiment, the SARM is a prodrug of the compound of formula VI. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula VI.

In another embodiment, the SARM compound that is effective at treating, preventing, suppressing, inhibiting or reducing the incidence of the ADIF-associated condition is a compound represented by the structure of formula VII.

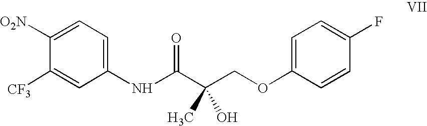

In one embodiment, the SARM is an analog of the compound of formula VII. In another embodiment, the SARM is a derivative of the compound of formula VII. In another embodiment, the SARM is an isomer of the compound of formula VII. In another embodiment, the SARM is a metabolite of the compound of formula VII. In another embodiment, the SARM is a pharmaceutically acceptable salt of the compound of formula VII. In another embodiment, the SARM is a pharmaceutical product of the compound of formula VII. In another embodiment, the SARM is a hydrate of the compound of formula VII. In another embodiment, the SARM is an N-oxide of the compound of formula VII. In another embodiment, the SARM is a crystal of the compound of formula VII. In another embodiment, the SARM is a polymorph of the compound of formula VII. In another embodiment, the SARM is a prodrug of the compound of formula VII. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula VII.

In another embodiment, the SARM compound that is effective at treating, preventing, suppressing, inhibiting or reducing the incidence of the ADIF-associated condition is a compound represented by the structure of formula VIII.

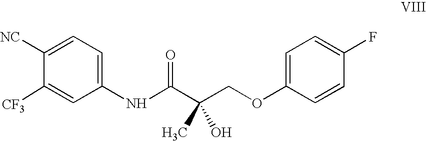

In one embodiment, the SARM is an analog of the compound of formula VIII. In another embodiment, the SARM is a derivative of the compound of formula VIII. In another embodiment, the SARM is an isomer of the compound of formula VIII. In another embodiment, the SARM is a metabolite of the compound of formula VIII. In another embodiment, the SARM is a pharmaceutically acceptable salt of the compound of formula VIII. In another embodiment, the SARM is a pharmaceutical product of the compound of formula VIII. In another embodiment, the SARM is a hydrate of the compound of formula VIII. In another embodiment, the SARM is an N-oxide of the compound of formula VIII. In another embodiment, the SARM is a crystal of the compound of formula VIII. In another embodiment, the SARM is a polymorph of the compound of formula VIII. In another embodiment, the SARM is a prodrug of the compound of formula VIII. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula VIII.

In another embodiment, the SARM compound that is effective at treating, preventing, suppressing, inhibiting or reducing the incidence of the ADIF-associated condition is a compound represented by the structure of formula IX.

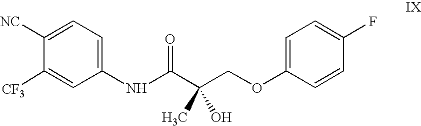

In one embodiment, the SARM is an analog of the compound of formula IX. In another embodiment, the SARM is a derivative of the compound of formula IX. In another embodiment, the SARM is an isomer of the compound of formula IX. In another embodiment, the SARM is a metabolite of the compound of formula IX. In another embodiment, the SARM is a pharmaceutically acceptable salt of the compound of formula IX. In another embodiment, the SARM is a pharmaceutical product of the compound of formula IX. In another embodiment, the SARM is a hydrate of the compound of formula IX. In another embodiment, the SARM is an N-oxide of the compound of formula IX. In another embodiment, the SARM is a crystal of the compound of formula IX. In another embodiment, the SARM is a polymorph of the compound of formula IX. In another embodiment, the SARM is a prodrug of the compound of formula IX. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula IX.

In another embodiment, the SARM compound that is effective at treating, preventing, suppressing, inhibiting or reducing the incidence of the ADIF-associated condition is a compound represented by the structure of formula X.

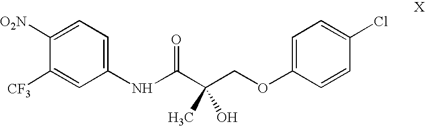

In one embodiment, the SARM is an analog of the compound of formula X. In another embodiment, the SARM is a derivative of the compound of formula X. In another embodiment, the SARM is an isomer of the compound of formula X. In another embodiment, the SARM is a metabolite of the compound of formula X. In another embodiment, the SARM is a pharmaceutically acceptable salt of the compound of formula X. In another embodiment, the SARM is a pharmaceutical product of the compound of formula X. In another embodiment, the SARM is a hydrate of the compound of formula X. In another embodiment, the SARM is an N-oxide of the compound of formula X. In another embodiment, the SARM is a crystal of the compound of formula X. In another embodiment, the SARM is a polymorph of the compound of formula X. In another embodiment, the SARM is a prodrug of the compound of formula X. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula X.

In another embodiment, the SARM compound that is effective at treating, preventing, suppressing, inhibiting or reducing the incidence of the ADIF-associated condition is a compound represented by the structure of formula M.

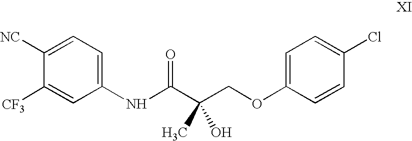

In one embodiment, the SARM is an analog of the compound of formula XI. In another embodiment, the SARM is a derivative of the compound of formula XI. In another embodiment, the SARM is an isomer of the compound of formula XI. In another embodiment, the SARM is a metabolite of the compound of formula XI. In another embodiment, the SARM is a pharmaceutically acceptable salt of the compound of formula XI. In another embodiment, the SARM is a pharmaceutical product of the compound of formula XI. In another embodiment, the SARM is a hydrate of the compound of formula XI. In another embodiment, the SARM is an N-oxide of the compound of formula XI. In another embodiment, the SARM is a crystal of the compound of formula XI. In another embodiment, the SARM is a polymorph of the compound of formula XI. In another embodiment, the SARM is a prodrug of the compound of formula XI. In another embodiment, the SARM is a combination of any of an analog, derivative, metabolite, isomer, pharmaceutically acceptable salt, pharmaceutical product, hydrate, N-oxide, crystal, polymorph or prodrug of the compound of formula XI.

The substituent R is defined herein as an alkyl, haloalkyl, dihaloalkyl, trihaloalkyl, CH₂F, CHF₂, CF₃, CF₂CF₃; aryl, phenyl, F, Cl, Br, I, alkenyl, or hydroxyl (OH).

An “alkyl” group refers to a saturated aliphatic hydrocarbon, including straight-chain, branched-chain and cyclic alkyl groups. In one embodiment, the alkyl group has 1-12 carbons. In another embodiment, the alkyl group has 1-7 carbons. In another embodiment, the alkyl group has 1-6 carbons. In another embodiment, the alkyl group has 1-4 carbons. The alkyl group may be unsubstituted or substituted by one or more groups selected from halogen (e.g. F, Cl, Br, I), hydroxy, alkoxy carbonyl, amido, alkylamido, dialkylamido, nitro, amino, alkylamino, dialkylamino, carboxyl, thio and thioalkyl.

A “haloalkyl” group refers to an alkyl group as defined above, which is substituted by one or more halogen atoms, e.g. by F, Cl, Br or I. A “halogen” refers to elements of Group VII or the periodic table, e.g. F, Cl, Br or I.

An “aryl” group refers to an aromatic group having at least one carbocyclic aromatic group or heterocyclic aromatic group, which may be unsubstituted or substituted by one or more groups selected from halogen (e.g. F, Cl, Br, I), haloalkyl, hydroxy, alkoxy carbonyl, amido, alkylamido, dialkylamido, nitro, amino, alkylamino, dialkylamino, carboxy or thio or thioalkyl. Nonlimiting examples of aryl rings are phenyl, naphthyl, pyranyl, pyrrolyl, pyrazinyl, pyrimidinyl, pyrazolyl, pyridinyl, furanyl, thiophenyl, thiazolyl, imidazolyl, isoxazolyl, and the like.

A “hydroxyl” group refers to an OH group. An “alkenyl” group refers to a group having at least one carbon to carbon double bond.

An “arylalkyl” group refers to an alkyl bound to an aryl, wherein alkyl and aryl are as defined above. An example of an aralkyl group is a benzyl group.

As contemplated herein, the present invention relates to the use of a SARM compound and/or its analog, derivative, isomer, metabolite, pharmaceutically acceptable salt, pharmaceutical product, hydrate, N-oxide, prodrug, polymorph or crystal or combinations thereof. In one embodiment, the invention relates to the use of an analog of the SARM compound. In another embodiment, the invention relates to the use of a derivative of the SARM compound. In another embodiment, the invention relates to the use of an isomer of the SARM compound. In another embodiment, the invention relates to the use of a metabolite of the SARM compound. In another embodiment, the invention relates to the use of a pharmaceutically acceptable salt of the SARM compound. In another embodiment, the invention relates to the use of a pharmaceutical product of the SARM compound. In another embodiment, the invention relates to the use of a hydrate of the SARM compound. In another embodiment, the invention relates to the use of an N-oxide of the SARM compound. In another embodiment, the invention relates to the use of a prodrug of the SARM compound. In another embodiment, the invention relates to the use of a polymorph of the SARM compound. In another embodiment, the invention relates to the use of a crystal of the SARM compound. In another embodiment, the invention relates to the use of any of a combination of an analog, derivative, isomer, metabolite, pharmaceutically acceptable salt, pharmaceutical product, hydrate, or N-oxide, prodrug, polymorph or crystal of the SARM compounds of the present invention.

As defined herein, the term “isomer” includes, but is not limited to, optical isomers and analogs, structural isomers and analogs, conformational isomers and analogs, and the like.

In one embodiment, this invention encompasses the use of various optical isomers of the SARM compounds. It will be appreciated by those skilled in the art that the SARM compounds of the present invention contain at least one chiral center. Accordingly, the SARM compounds used in the methods of the present invention may exist in, and be isolated in, optically-active or racemic forms. Some compounds may also exhibit polymorphism. It is to be understood that the present invention encompasses any racemic, optically-active, polymorphic, or stereroisomeric form, or mixtures thereof, which form possesses properties useful in the treatment of obesity and related disorders as described herein. In one embodiment, the SARM compounds are the pure (R)-isomers. In another embodiment, the SARM compounds are the pure (S)-isomers. In another embodiment, the SARM compounds are a mixture of the (R) and the (S) isomers. In another embodiment, the SARM compounds are a racemic mixture comprising an equal amount of the (R) and the (S) isomers. It is well known in the art how to prepare optically-active forms (for example, by resolution of the racemic form by recrystallization techniques, by synthesis from optically-active starting materials, by chiral synthesis, or by chromatographic separation using a chiral stationary phase).

The invention includes pharmaceutically acceptable salts of amino-substituted compounds with organic and inorganic acids, for example, citric acid and hydrochloric acid. The invention also includes N-oxides of the amino substituents of the compounds described herein. Pharmaceutically acceptable salts can also be prepared from the phenolic compounds by treatment with inorganic bases, for example, sodium hydroxide. Also, esters of the phenolic compounds can be made with aliphatic and aromatic carboxylic acids, for example, acetic acid and benzoic acid esters.

This invention further includes derivatives of the SARM compounds. The term “derivatives” includes but is not limited to ether derivatives, acid derivatives, amide derivatives, ester derivatives and the like. In addition, this invention further includes hydrates of the SARM compounds. The term “hydrate” includes but is not limited to hemihydrate, monohydrate, dihydrate, trihydrate and the like.

This invention further includes pharmaceutical products of the SARM compounds. The term “pharmaceutical product” means a composition suitable for pharmaceutical use (pharmaceutical composition), as defined herein.

This invention further includes prodrugs of the SARM compounds. The term “prodrug” means a substance which can be converted in-vivo into a biologically active agent by such reactions as hydrolysis, esterification, desterification, activation, salt formation and the like.

This invention further includes crystals of the SARM compounds. Furthermore, this invention provides polymorphs of the SARM compounds. The term “crystal” means a substance in a crystalline state. The term “polymorph” refers to a particular crystalline state of a substance, having particular physical properties such as X-ray diffraction, IR spectra, melting point, and the like.

Biological Activity of Selective Androgen Modulator Compounds

Selective androgen receptor modulator (SARM) compounds are a novel class of androgen receptor targeting agents (“ARTA”). Several appropriately substituted SARM compounds demonstrate androgenic and anabolic activity of a nonsteroidal ligand for the androgen receptor. SARM compounds have previously been shown to be useful for a) male contraception; b) treatment of a variety of hormone-related conditions, for example conditions associated with Androgen Decline in Aging Male (ADAM), such as sexual dysfunction, decreased sexual libido, erectile dysfunction, hypogonadism, sarcopenia, osteopenia, osteoporosis, alterations in cognition and mood, depression, anemia, muscle weakness, hair loss, obesity, benign prostate hyperplasia and prostate cancer; c) treatment of conditions associated with Androgen Decline in Female (ADIF), such as sexual dysfunction, decreased sexual libido, hypogonadism, sarcopenia, osteopenia, osteoporosis, alterations in cognition and mood, depression, anemia, hair loss, obesity and endometriosis, d) treatment and/or prevention of chronic muscular wasting; e) decreasing the incidence of, halting or causing a regression of prostate cancer; f) oral androgen replacement; g) preventing and/or treating dry eye conditions; h) treatment and/or prevention of benign prostate hyperplasia (BPH); i) inducing apoptosis in a cancer cell; j) treatment and/or prevention of cancers of female sexual organs such as breast cancer, uterine cancer and ovarian cancer; and/or other clinical therapeutic and/or diagnostic areas.

These novel agents are useful in males for the treatment of a variety of hormone-related conditions including ADIF-associated conditions, such Further, SARMs are useful for oral testosterone replacement therapy, and imaging prostate cancer.

As contemplated herein, the SARM compounds of the present invention as useful in treating, preventing, suppressing, inhibiting or reducing the incidence of an Androgen Deficiency in Female (ADIF)-associated condition in a female subject, including but not limited to sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and ovarian cancer.

In one embodiment, the ADIF-associated condition is sexual dysfunction. In another embodiment, the ADIF-associated condition is decreased sexual libido. The term “libido, as used herein, means sexual desire.

In another embodiment, the ADIF-associated condition is hypogonadism. “Hypogonadism” is a condition resulting from or characterised by abnormally decreased functional activity of the gonads, with retardation of growth and sexual development.

In another embodiment, the ADIF-associated condition is sarcopenia. In another embodiment, the ADIF-associated condition is osteopenia. “Osteopenia” refers to decreased calcification or density of bone. This is a term which encompasses all skeletal systems in which such a condition is noted.

In another embodiment, the ADIF-associated condition is osteoporosis. “Osteoporosis” refers to a thinning of the bones with reduction in bone mass due to depletion of calcium and bone protein. Osteoporosis predisposes a person to fractures, which are often slow to heal and heal poorly. Unchecked osteoporosis can lead to changes in posture, physical abnormality, and decreased mobility.

In another embodiment, the ADIF-associated condition is associated with an alternation in cognition and mood. The term “cognition” refers to the process of knowing, specifically the process of being aware, knowing, thinking, learning and judging. Cognition is related to the fields of psychology, linguistics, computer science, neuroscience, mathematics, ethology and philosophy. The term “mood” refers to a temper or state of the mind. As contemplated herein, alterations means any change for the positive or negative, in cognition and/or mood.

In another embodiment, the ADIF-associated condition is depression. The term “depression” refers to an illness that involves the body, mood and thoughts, that affects the way a person eats, sleeps and the way one feels about oneself, and thinks about things.

The signs and symptoms of depression include loss of interest in activities, loss of appetite or overeating, loss of emotional expression, an empty mood, feelings of hopelessness, pessimism, guilt or helplessness, social withdrawal, fatigue, sleep disturbances, trouble concentrating, remembering, or making decisions, restlessness, irritability, headaches, digestive disorders or chronic pain.

In another embodiment, the ADIF-associated condition is fatigue. The term “fatigue” refers to a state characterized by a lessened capacity for work and reduced efficiency of accomplishment, usually accompanied by a feeling of weariness and tiredness.

In another embodiment, the ADIF-associated condition is hair loss. The term “hair loss”, medically known as alopecia, refers to baldness as in the very common type of male-pattern baldness. Baldness typically begins with patch hair loss on the scalp and sometimes progresses to complete baldness and even loss of body hair. Hair loss affects both males and females.

In another embodiment, the ADIF-associated condition is anemia. “Anemia” refers to the condition of having less than the normal number of red blood cells or less than the normal quantity of hemoglobin in the blood. The oxygen-carrying capacity of the blood is, therefore, decreased. Persons with anemia may feel tired and fatigue easily, appear pale, develop palpitations and become usually short of breath. Anemia is caused by four basic factors: a) hemorrhage (bleeding); b) hemolysis (excessive destruction of red blood cells); c) underproduction of red blood cells; and d) not enough normal hemoglobin. There are many forms of anemia, including aplastic anemia, benzene poisoning, Fanconi anemia, hemolytic disease of the newborn, hereditary spherocytosis, iron deficiency anemia, osteopetrosis, pernicious anemia, sickle cell disease, thalassemia, myelodysplastic syndrome, and a variety of bone marrow diseases. As contemplated herein, the SARM compounds of the present invention are useful in preventing and/or treating any one or more of the above-listed forms of anemia.

In another embodiment, the ADIF-associated condition is obesity. “Obesity” refers to the state of being well above one's normal weight. Traditionally, a person is considered to be obese if they are more than 20 percent over their ideal weight. Obesity has been more precisely defined by the National Institute of Health (NIH) as a Body to Mass Index (BMI) of 30 or above. Obesity is often multifactorial, based on both genetic and behavioral factors. Overweight due to obesity is a significant contributor to health problems. It increases the risk of developing a number of diseases including: Type 2 (adult-onset) diabetes; high blood pressure (hypertension); stroke (cerebrovascular accident or CVA); heart attack (myocardial infarction or M); heart failure (congestive heart failure); cancer (certain forms such as cancer of the prostate and cancer of the colon and rectum); gallstones and gallbladder disease (cholecystitis); Gout and gouty arthritis; osteoarthritis (degenerative arthritis) of the knees, hips, and the lower back; sleep apnea (failure to breath normally during sleep, lowering blood oxygen); and Pickwickian syndrome (obesity, red face, underventilation and drowsiness). As contemplated herein, the term “obesity” includes any one of the above-listed obesity-related conditions and diseases. Thus the SARM compounds of the present invention are useful in preventing and/or treating obesity and any one or more of the above-listed obesity-related conditions and diseases.

In another embodiment, the ADIF-associated condition is polycystic ovarian disease. The term “polycystic ovarian disease” refers to a condition found among women who do not ovulate, characterized by multiple ovarian cysts and increased androgen production.

In another embodiment, the ADIF-associated condition is muscle weakness. The term “muscle weakness”. The term “muscle weakness” refers to a broad spectrum complaint of debility, fatigue or exhastion attributable to weakness of various muscles. The weakness can be characterized as subacute or chronic, often progressive, and is a manifestation of many muscle and neuromuscular disease.

In another embodiment, the ADIF-associated condition is endometriosis. The term “endometriosis” refers to a condition wherein cells that normally grow inside the uterus (womb), instead grow outside the uterus. The common symptoms and signs are pain (usually pelvic) and infertility. Pelvic pain usually occurs during or just before menstruation and lessens after menstruation. Some women experience pain or cramping with intercourse, bowel movements and/or urination. Endometriosis can cause infertility. It believed that endometriosis bleeding, inflammation, and scarring can cause distortion of the female reproductive organs (such as obstruction of the fallopian tubes), resulting in infertility. Other symptoms related to endometriosis include lower abdominal pain, diarrhea or constipation, low back pain, irregular or heavy menstrual bleeding, or even blood in the urine. Rare symptoms of endometriosis include chest pain or coughing blood due to endometriosis in the lungs, headache and/or seizures due to endometriosis in the brain. Endometriosis can become cancerous in less than 1% of women. Most of the cancers found with the condition, however, appear not to be associated with the implants, but rather occur independently of the disease.

In another embodiment, the ADIF-associated condition is breast cancer. The term “breast cancer” refers to a group of breast malignancies that differ in their capability of spreading to other body tissues (metastasis).

In another embodiment, the ADIF-associated condition is uterine cancer. The term “uterine cancer” refers to a cancer of the uterine (womb). Cancer of the uterus occurs most often in women between the ages of 55 and 70 years. Abnormal bleeding after menopause is the most common symptom of cancer of the uterus. Cancer of the uterus is diagnosed based on the results of the pelvic examination, pap smear, biopsy of the uterus, and D and C procedure.

In another embodiment, the ADIF-associated condition is ovarian cancer. The term “ovarian cancer” refers to a cancer of the ovary, the egg sac of females. There are several types of ovarian cancer. Ovarian cancer that begins on the surface of the ovary (epithelial carcinoma) is the most common type. Women who have a family history of ovarian cancer are at an increased risk of developing ovarian cancer. Hereditary ovarian cancer makes up approximately 5 to 10% of all cases of ovarian cancer. Three hereditary patterns have been identified: ovarian cancer alone, ovarian and breast cancers, and ovarian and colon cancers. Ovarian cancer is hard to detect early because there usually are no symptoms and those symptoms that do occur tend to be vague. Detection of ovarian cancer involves physical examination (including pelvic exam), ultrasound, x-ray tests,CA-125 blood test and biopsy of the ovary.

In one embodiment, the female subject, which the SARM compounds of the present invention are administered to is an aging female subject. As defined herein, the term “aging” means a process of becoming older. In one embodiment, the aging female is a female over 40 years old. In another embodiment, the aging female is a female over 45 years old. In another embodiment, the aging female is a female over 45 years old. In another embodiment, the aging female is a female over 50 years old. In another embodiment, the aging female is a female over 55 years old. In another embodiment, the aging female is a female over 60 years old. In another embodiment, the aging female is a female over 65 years old. In another embodiment, the aging female is a female over 70 years old. In another embodiment, the aging female is a female over 75 years old.

As contemplated herein, the SARM compounds of the present invention are effective at treating or preventing different ADIF-associated conditions, and may be categorized into subgroups depending on their biological activity. For example, several SARM compounds have an agonistic effect on muscle or bone. Other SARM compounds have no effect on muscle or bone. Other SARM compounds are able to penetrate the central nervous system (CNS). Other SARM compounds do not penetrate the central nervous system (CNS).

The SARM compounds of the present invention are a novel class of androgen receptor targeting agents (ARTA) which demonstrate androgenic or antiandrogenic and anabolic activity of a nonsteroidal ligand for the androgen receptor. The agents define a new subclass of compounds, which are selective androgen receptor modulators (SARMs).

The androgen receptor (AR) is a ligand-activated transcriptional regulatory protein that mediates induction of male sexual development and function through its activity with endogenous androgens (male sex hormones). The androgenic hormones are steroids which are produced in the body by the testis and the cortex of the adrenal gland.

Androgenic steroids play an important role in many physiologic processes, including the development and maintenance of male sexual characteristics such as muscle and bone mass, prostate growth, spermatogenesis, and the male hair pattern (Matsumoto, Endocrinol. Met. Clin. N. Am. 23:857-75 (1994)). The endogenous steroidal androgens include testosterone and dihydrotestosterone (“DHT”). Other steroidal androgens include esters of testosterone, such as the cypionate, propionate, phenylpropionate, cyclopentylpropionate, isocarporate, enanthate, and decanoate esters, and other synthetic androgens such as 7-Methyl-Nortestosterone (“MENT”) and its acetate ester (Sundaram et al., “7 Alpha-Methyl-Nortestosterone(MENT): The Optimal Androgen For Male Contraception,” Ann. Med., 25:199-205 (1993) (“Sundaram”)).

As contemplated herein, this invention provides a class of compounds which are Selective Androgen Receptor Modulator (SARM) compounds. These compounds, which are useful in preventing and treating ADIF-associated conditions are classified as androgen receptor agonists (AR agonists), partial agonists or androgen receptor antagonists (AR antagonists).

A receptor agonist is a substance which binds receptors and activates them. A receptor partial agonist is a substance, which binds receptor and partially activate them. A receptor antagonist is a substance which binds receptors and inactivates them. As demonstrated herein, the SARM compounds of the present invention have a tissue-selective effect, wherein one agent may be an agonist, partial agonist and/or antagonist, depending on the tissue. For example, the SARM compound may stimulate muscle tissue and at the same time inhibit prostate tissue. In one embodiment, the SARMs which are useful in treating and preventing ADIF-associated conditions are AR agonists, and are, therefore, useful in binding to and activating the AR. In another embodiment, the SARMs which are useful in treating and preventing ADIF-associated conditions are AR antagonists, and are, therefore, useful in binding to and inactivating the AR. Assays to determine whether the compounds of the present invention are AR agonists or antagonists are well known to a person skilled in the art. For example, AR agonistic activity can be determined by monitoring the ability of the SARM compounds to maintain and/or stimulate the growth of AR containing tissue such as prostate and seminal vesicles, as measured by weight. AR antagonistic activity can be determined by monitoring the ability of the SARM compounds inhibit the growth of AR containing tissue.

In yet another embodiment, the SARM compounds of the present invention can be classified as partial AR agonist/antagonists. The SARMs are AR agonists in some tissues, to cause increased transcription of AR-responsive genes (e.g. muscle anabolic effect). In other tissues, these compounds serve as competitive inhibitors of testosterone/DHT on the AR to prevent agonistic effects of the native androgens.

The SARM compounds of the present invention bind either reversibly or irreversibly to the androgen receptor. In one embodiment, the SARM compounds bind reversibly to the androgen receptor. In another embodiment, the SARM compounds bind irreversibly to the androgen receptor. The compounds of the present invention may contain a functional group (affinity label) that allows alkylation of the androgen receptor (i.e. covalent bond formation). Thus, in this case, the compounds bind irreversibly to the receptor and, accordingly, cannot be displaced by a steroid, such as the endogenous ligands DHT and testosterone.

Pharmaceutical Compositions

As used herein, “pharmaceutical composition” means a “therapeutically effective amount” of the active ingredient, i.e. the SARM compound, together with a pharmaceutically acceptable carrier or diluent. A “therapeutically effective amount” as used herein refers to that amount which provides a therapeutic effect for a given condition and administration regimen.

The pharmaceutical compositions containing the SARM agent can be administered to a subject by any method known to a person skilled in the art, such as parenterally, paracancerally, transmucosally, transdermally, intramuscularly, intravenously, intradermally, subcutaneously, intraperitonealy, intraventricularly, intracranially, intravaginally or intratumorally.

In one embodiment, the pharmaceutical compositions are administered orally, and are thus formulated in a form suitable for oral administration, i.e. as a solid or a liquid preparation. Suitable solid oral formulations include tablets, capsules, pills, granules, pellets and the like. Suitable liquid oral formulations include solutions, suspensions, dispersions, emulstions, oils and the like. In one embodiment of the present invention, the SARM compounds are formulated in a capsule. In accordance with this embodiment, the compositions of the present invention comprise in addition to the SARM active compound and the inert carrier or diluent, a hard gelating capsule.

Further, in another embodiment, the pharmaceutical compositions are administered by intravenous, intraarterial, or intramuscular injection of a liquid preparation. Suitable liquid formulations include solutions, suspensions, dispersions, emulsions, oils and the like. In one embodiment, the pharmaceutical compositions are administered intravenously, and are thus formulated in a form suitable for intravenous administration. In another embodiment, the pharmaceutical compositions are administered intraarterially, and are thus formulated in a form suitable for intraarterial administration. In another embodiment, the pharmaceutical compositions are administered intramuscularly, and are thus formulated in a form suitable for intramuscular administration.

Further, in another embodiment, the pharmaceutical compositions are administered topically to body surfaces, and are thus formulated in a form suitable for topical administration. Suitable topical formulations include gels, ointments, crearns, lotions, drops and the like. For topical administration, the SARM agents or their physiologically tolerated derivatives such as salts, esters, N-oxides, and the like are prepared and applied as solutions, suspensions, or emulsions in a physiologically acceptable diluent with or without a pharmaceutical carrier.

Further, in another embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of SARM agent over a period of time.

In another embodiment, the active compound can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 317-327; see generally ibid).

As used herein “pharmaceutically acceptable carriers or diluents” are well known to those skilled in the art. The carrier or diluent may be a solid carrier or diluent for solid formuations, a liquid carrier or diluent for liquid formulations, or mixtures thereof.

Solid carriers/diluents include, but are not limited to, a gum, a starch (e.g. corn starch, pregeletanized starch), a sugar (e.g., lactose, mannitol, sucrose, dextrose), a cellulosic material (e.g. microcrystalline cellulose), an acrylate (e.g. polymethylacrylate), calcium carbonate, magnesium oxide, talc, or mixtures thereof.

For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

Parenteral vehicles (for subcutaneous, intravenous, intraarterial, or intramuscular injection) include sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Examples are sterile liquids such as water and oils, with or without the addition of a surfactant and other pharmaceutically acceptable adjuvants. In general, water, saline, aqueous dextrose and related sugar solutions, and glycols such as propylene glycols or polyethylene glycol are preferred liquid carriers, particularly for injectable solutions. Examples of oils are those of petroleum, animal, vegetable, or synthetic origin, for example, peanut oil, soybean oil, mineral oil, olive oil, sunflower oil, and fish-liver oil.

In addition, the compositions may further comprise binders (e.g. acacia, cornstarch, gelatin, carbomer, ethyl cellulose, guar gum, hydroxypropyl cellulose, hydroxypropyl methyl cellulose, povidone), disintegrating agents (e.g. cornstarch, potato starch, alginic acid, silicon dioxide, croscarmelose sodium, crospovidone, guar gum, sodium starch glycolate), buffers (e.g., Tris-HCI., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., glycerol, polyethylene glycerol), anti-oxidants (e.g., ascorbic acid, sodium metabisulfite, butylated hydroxyanisole), stabilizers (e.g. hydroxypropyl cellulose, hyroxypropylnethyl cellulose), viscosity increasing agents(e.g. carbomer, colloidal silicon dioxide, ethyl cellulose, guar gum), sweeteners (e.g. aspartame, citric acid), preservatives (e.g., Thimerosal, benzyl alcohol, parabens), lubricants (e.g. stearic acid, magnesium stearate, polyethylene glycol, sodium lauryl sulfate), flow-aids (e.g. colloidal silicon dioxide), plasticizers (e.g. diethyl phthalate, triethyl citrate), emulsifiers (e.g. carbomer, hydroxypropyl cellulose, sodium lauryl sulfate), polymer coatings (e.g., poloxamers or poloxarnines), coating and film forming agents (e.g. ethyl cellulose, acrylates, polymethacrylates) and/or adjuvants.

In one embodiment, the pharmaceutical compositions provided herein are controlled release compositions, i.e. compositions in which the SARM compound is released over a period of time after administration. Controlled or sustained release compositions include formulation in lipophilic depots (e.g. fatty acids, waxes, oils). In another embodiment, the composition is an immediate release composition, i.e. a composition in which all of the SARM compound is released immediately after administration.

In yet another embodiment, the pharmaceutical composition can be delivered in a controlled release system. For example the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In yet another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp.115-138 (1984). Other controlled release systems are discussed in the review by Langer (Science 249:1527-1533 (1990).

The compositions may also include incorporation of the active material into or onto particulate preparations of polymeric compounds such as polylactic acid, polglycolic acid, hydrogels, etc, or onto liposomes, microemulsions, micelles, unilameflar or multilamellar vesicles, erythrocyte ghosts, or spheroplasts.) Such compositions will influence the physical state, solubility, stability, rate of in vivo release, and rate of in vivo clearance.

Also comprehended by the invention are particulate compositions coated with Polymers (e.g. poloxamers or poloxamines) and the compound coupled to antibodies directed,.agaiinst tissue-specific receptors, ligands or antigens or coupled to ligands of tissue-specific receptors.

Also comprehended by the invention are compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline. The modified compounds are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The preparation of pharmaceutical compositions which contain an active component is well understood in the art, for example by mixing, granulating, or tablet-forming processes. The active therapeutic ingredient is often mixed with excipients which are pharmaceutically acceptable and compatible with the active ingredient. For oral administration, the SARM agents or their physiologically tolerated derivatives such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. For parenteral administration, the SARM agents or their physiologically tolerated derivatives such as salts, esters, N-oxides, and the like are converted into a solution, suspension, or emulsion, if desired with the substances customary and suitable for this purpose, for example, solubilizers or other.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from-inorganic bases such as, for example, sodium, potassium, ammonium, calcium, or ferric hydroxides, and such organic bases as isopropylamine, trimethylamine, 2-ethylamino ethanol, histidine, procaine, and the like.

For use in medicine, the salts of the SARM will be pharmaceutically acceptable salts. Other salts may, however, be useful in the preparation of the compounds according to the invention or of their pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, sulphuric acid, methanesuiphonic acid, fumaric acid, maleic acid, succinic acid, acetic acid, benzoic: acid, oxalic acid, citric acid, tartaric acid, carbonic acid or phosphoric acid.

As defined herein, “contacting” means that the SARM compound of the present invention is introduced into a sample containing the enzyme in a test tube, flask, tissue culture, chip, array, plate, microplate, capillary, or the like, and incubated at a temperature and time sufficient to permit binding of the SARM to the enzyme. Methods for contacting the samples with the SARM or other specific binding components are known to those skilled in the art and may be selected depending on the type of assay protocol to be run. Incubation methods are also standard and are known to those skilled in the art.

In another embodiment, the term “contacting” means that the SARM compound of the present invention is introduced into a subject receiving treatment, and the SARM compound is allowed to come in contact with the androgen receptor in vivo.

As used herein, the term “treating” includes preventative as well as disorder remitative treatment. As used herein, the terms “reducing”, “suppressing” and “inhibiting” have their commonly understood meaning of lessening or decreasing. As used herein, the term “progression” means increasing in scope or severity., advancing, growing or becoming worse. As used herein, the term “recurrence” means the return of a disease after a remission.

As used herein, the term “administering” refers to bringing a subject in contact with a SARM compound of the present invention. As used herein, administration can be accomplished in vitro, i.e. in a test tube, or in vivo, i.e. in cells or tissues of living organisms, for example humans. In one embodiment, the present invention encompasses administering the compounds of the present invention to a subject.

In one embodiment, the methods of the present invention comprise administering a SARM compound as the sole active ingredient. However, also encompassed within the scope of the present invention are methods for treating and/or preventing ADIF-associated conditions, which comprise administering the SARM compounds in combination with one or more therapeutic agents. These agents include, but are not limited to: LHRH analogs, reversible antiandrogens, antiestrogens, anticancer drugs, 5-alpha reductase inhibitors, aromatase inhibitors, progestins, agents acting through other nuclear hormone receptors, selective estrogen receptor modulators (SERM), progesterone, estrogen, PDE5 inhibitors, apomorphine, bisphosphonate, and one or more additional SARMS.

Thus, in one embodiment, the methods of the present invention comprise administering the selective androgen receptor modulator compound, in combination with an LHRH analog. In another embodiment, the methods of the present invention comprise administering a selective androgen receptor modulator compound, in combination with a reversible antiandrogen. In another embodiment, the methods of the present invention comprise administering a selective androgen receptor modulator compound, in combination with an antiestrogen. In another embodiment, the methods of the present invention comprise administering a selective androgen receptor modulator compound, in combination with an anticancer drug. In another embodiment, the methods of the present invention comprise administering a selective androgen receptor modulator compound, in combination with a 5-alpha reductase inhibitor. In another embodiment, the methods of the present invention comprise administering a selective androgen receptor modulator compound, in combination with an aromatase inhibitor. In another embodiment, the methods of the present invention comprise administering a selective androgen receptor modulator compound, in combination with a progestin. In another embodiment, the methods of the present invention comprise administering a selective androgen receptor modulator compound, in combination with an agent acting through other nuclear hormone receptors. In another embodiment, the methods of the present invention comprise administering a selective androgen receptor modulator compound, in combination with a selective estrogen receptor modulators (SERM). In another embodiment, the methods of the present invention comprise administering a selective androgen receptor modulator compound, in combination with a progesterone. In another embodiment, the methods of the present invention comprise administering a selective androgen receptor modulator compound, in combination with an estrogen. In another embodiment, the methods of the present invention comprise administering a selective androgen receptor modulator compound, in combination with a PDE5 inhibitor. In another embodiment, the methods of the present invention comprise administering a selective androgen receptor modulator compound, in combination with apomorphine. In another embodiment, the methods of the present invention comprise administering a selective androgen receptor modulator compound, in combination with a bisphosphonate. In another embodiment, the methods of the present invention comprise administering a selective androgen receptor modulator compound, in combination with one or more additional SARMS.

The following examples are presented in order to more fully illustrate the preferred embodiments of the invention. They should in no way, however, be construed as limiting the broad scope of the invention.

### EXPERIMENTAL DETAILS SECTION

### EXAMPLE 1

### Effect of Compound VI on Myosin Heavy Chain (MHC) subtype IIb m-RNA Expression

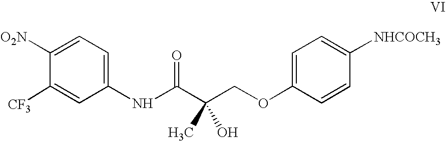 Methods:

To demonstrate the importance of Compound VI in muscle, Applicants have examined the effects of this nonsteroidal anabolic agent directly in skeletal muscle by monitoring the expression of myosin heavy chain (MHC) subtypes using RT-PCR. MHC is the predominant protein in skeletal muscle encoded by a multigene family expressed in a tissue-specific and developmentally regulated manner. In steady state, MRNA expression usually parallels the pattern of MHC protein expression. Because transcription of MHC MRNA occurs in advance of MHC protein translation, and the increased sensitivity of RT-PCR compared to western blotting, rapid changes in mRNA expression can be detected and used to analyze the subtle dynamic effects of muscle anabolism.

Results:

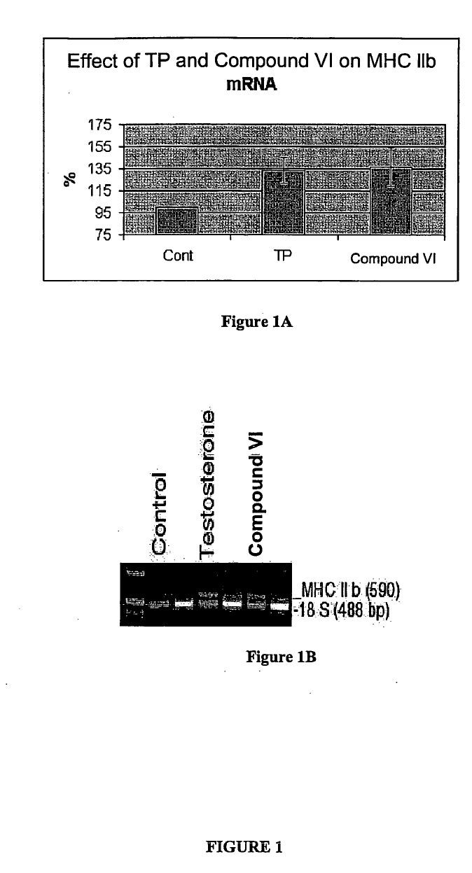

The masseter muscle dissected from untreated intact female rats was set as the control level (representing 100%) of MHC IIb expression (FIG. 1A). Intact female rats treated with androgens were evaluated against the untreated controls for the effect of treatment on MHC IIb from masseter. The results indicate that testosterone propionate has a positive effect on masseter muscle where it increased transcription of MHC type IIb to 133% of untreated control (FIG. 1A). Compound VI was also anabolic in muscle, with an increase in MHC type IIb to 137% (FIG. 1A). Actual untransformed PCR data is shown in FIG. 1B. The results indicate that treatment with compound VI results in increased muscle fiber RNA in females.

### EXAMPLE 2

### Effect of SARMS on BMC (Bone Mineral Content) and BMD (Bone Mineral Density) in Female Ovariectomized Rats

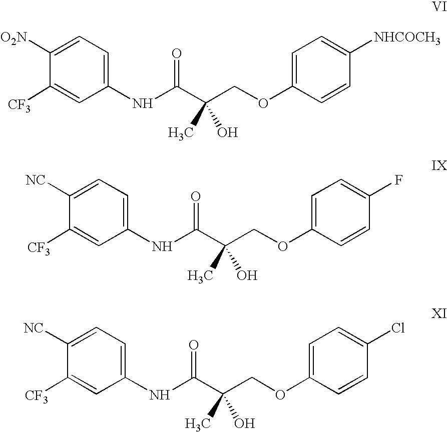

Two hundred and sixty (260) female Sprague-Dawley rats (23 weeks of age) were purchased from an approved vendor and used in this study. Animals were randomized (n=10 per group) into each of the treatment groups outlined in the table below. Animals assigned to groups 6 through 26 undergo surgical ovariectomy (OVX) on day 1 of the experiment. Drug administration with Compound VI, Compound IX and Compound XI, antiandrogen, and/or DHT began immediately (i.e., on the day that OVX was performed) or 90 days after OVX to assess the ability of these compounds to inhibit bone resorption (immediate treatment) or stimulate bone formation (delayed treatment). The compound of interest was administered via daily subcutaneous injection (0.25 mL) and continued until day 180 of the study. Drug solutions were prepared daily by dissolving in ethanol and dilution with polyethylene glycol 300. The percentage of ethanol was the same in all vehicles, and was determined based on the solubility of the test compounds. Whole body DEXA images were collected for up to 180 days after OVX, as described in the table below. Bone mineral density (BMD), bone mineral content (BMC), bone mineral area (BMA), lean body mass (LBM), fat mass (FM), total body mass (TBM), and sub-regional BMD in the lumbar vertebrae and left femur were determined at each time point. All animals were sacrificed on day 181. Femurs and tibias were excised from the sacrificed rats for future studies. Serum and urine specimens were collected prior to or at the time of sacrifice and used to determine serum concentrations of osteocalcin, IL-6, IGF-1, and urinary concentrations of deoxypyridinoline, and creatinine for 5 animals in each group.

The results are presented in Table 1:

TABLE 1

| Group | | Delayed Tx | Immediate Tx | Bicalutamide | DHT | Days on which DEXA |
|---|---|---|---|---|---|---|
| # | Status | (mg/day) | (mg/day) | (mg/day) | (mg/day) | will be performed |
| 1 | Intact | — | — | — | — | 0, 30, 60, 90, 120, 150, 180 |
| 2 | Intact | 1.0 (VI) | — | — | — | 0, 90, 120, 150, 180 |
| 3 | Intact | — | 1.0 (VI) | — | — | 0, 30, 60, 90 |
| 4 | Intact | — | — | — | 1.0 | 0, 90, 120, 150, 180 |
| 5 | Intact | — | — | — | 1.0 | 0, 30, 60, 90 |
| 6 | OVX | — | — | — | — | 0, 30, 60, 90, 120, 150, 180 |
| 7 | OVX | 0.10 (VI) | — | — | — | 0, 90, 120, 150, 180 |
| 8 | OVX | 0.30.(VI) | — | — | — | 0, 90, 120, 150, 180 |
| 9 | OVX | 0.50 (VI) | — | — | — | 0, 90, 120, 150, 180 |
| 10 | OVX | 0.75 (VI) | — | — | — | 0, 90, 120, 150, 180 |
| 11 | OVX | 1.00 (VI) | — | — | — | 0, 90, 120, 150, 180 |
| 12 | OVX | 3.00 (VI) | — | — | — | 0, 90, 120, 150, 180 |
| 13 | OVX | — | 0.10 (VI) | — | — | 0, 30, 60, 90 |
| 14 | OVX | — | 0.30 (VI) | — | — | 0, 30, 60, 90 |
| 15 | OVX | — | 0.50 (VI) | — | — | 0, 30, 60, 90 |
| 16 | OVX | — | 0.75 (VI) | — | — | 0, 30, 60, 90 |
| 17 | OVX | — | 1.00 (VI) | — | — | 0, 30, 60, 90 |
| 18 | OVX | — | 3.00 (VI) | — | — | 0, 30, 60, 90 |
| 19 | OVX | 0.5 (VI) | — | 1.0 | — | 0, 90, 120, 150, 180 |
| 20 | OVX | — | 0.5 (VI) | 1.0 | — | 0, 30, 60, 90 |
| 21 | OVX | — | — | — | 1.0 | 0, 30, 60, 90 |
| 22 | OVX | — | — | — | 1.0 | 0, 90, 120, 150, 180 |
| 23 | OVX | 1.00 (IX) | — | — | — | 0, 90, 120, 150, 180 |
| 24 | OVX | — | 1.00 (VI) | — | — | 0, 30, 60, 90 |
| 25 | OVX | 1.00 (XI) | — | — | — | 0, 90, 120, 150, 180 |
| 26 | OVX | — | 1.00 (XI) | — | — | 0, 30, 60, 90 |

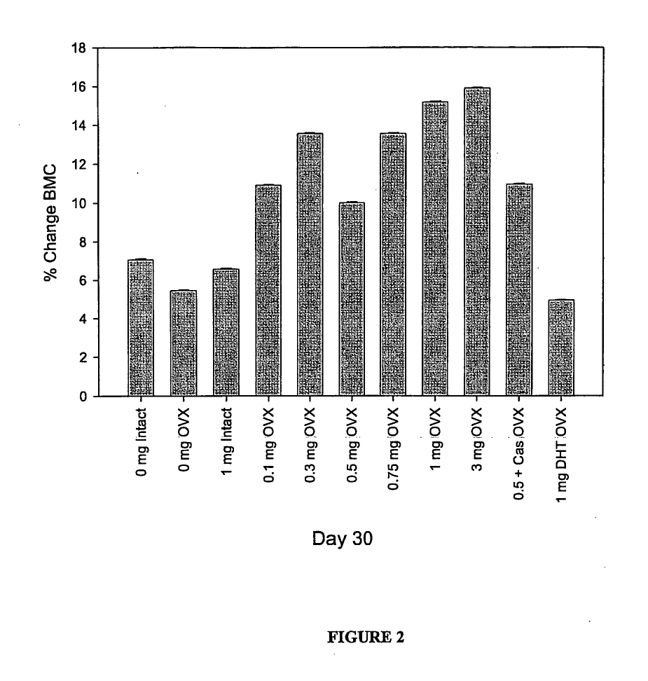

FIG. 2 illustrates the % change in bone mineral content from baseline over the 30 day treatment period following ovariectomy. Over the study period, the ovariectomized female rats treated with placebo alone exhibited a decreased bone mineral content of −1.8% relative to intact control females. Compound VI maintained and improved the bone mineral content when compared with the intact control or the placebo-treated groups. BMC was increased relative to the placebo-treated ovariectomized controls by approximately 5%, 8.3%, 4.7%, 8.3%, and 10% and 11.6% in animals treated with doses of 0.1, 0.3, 0.5, 0.75, 1, and 3 mg Compound VI, respectively. These results demonstrate the direct positive anabolic effect of Compound VI on the skeletal system of ovariectomized female rats and indicate that Compound VI will be a significant therapeutic tool for preventing and/or treating osteoporosis in elderly female subjects. Of particular interest is the potential application of Compound VI for hormone replacement therapy in females that are afflicted by androgen deficiency.

### EXAMPLE 3

### Bone Sparing Effects of a Selective Androgen Receptor Modulator

S-3-(4-acetylamino-phenoxy)-2-hydroxy-2-methyl-N-(4-nitro-3-trifluoromethyl-phenyl)-propionamide (Compound VI) is a Selective Androgen Receptor Modulator that has potent binding affinity for the Androgen Receptor (AR) (Kᵢ=4.0±0.7 nM), and that exhibits tissue-selective androgenic and anabolic effects in rats. In castrated male rats, S-4 showed dose-dependent effects in the levator ani muscle. These effects were similar in potency and efficacy to those of testosterone propionate (TP). However, S-4 was only a partial agonist in the prostate and seminal vesicles, restoring them to 34% and 28% of intact animals, respectively. Since S-4 exerts tissue specific anabolic effects, it may be an ideal compound to elucidate the effects of androgens on the female skeleton. The purpose of these studies was to evaluate the protective effects of S-4 in an ovariectomized (OVX) rat model of postmenopausal bone loss.

Materials and Methods:

Animals:

One hundred twenty female Sprague-Dawley rats were purchased from Harlan (Indiapolis, Ind.). The animals were housed three per cage and were allowed free access to tap water and commercial rat chow (Harlan Teklad 22/5 rodent diet—8640). During the course of the study, the animals were maintained on a 12 hr light:dark cycle. This study was reviewed and approved by the Institutional Laboratory Care and Use Committee of The Ohio State University. At 23 weeks of age, the animals were randomly assigned to one of 12 treatment groups of 10 animals as follows: (1) OVX+Compound VI (0.1 mg/day); (2) OVX+Compound VI (0.3 mg/day); (3) OVX+Compound VI (0.5 mg/day); (4) OVX+Compound VI (0.75 mg/day); (5) OVX+Compound VI (1.0 mg/day); (6) OVX+Compound VI (3.0 mg/day); (7) OVX+DHT (1 mg/day); (8) OVX+Compound VI+Antiandrogen (0.5±1.0 mg/day); (9) OVX+Vehicle; (10) intact+Compound VI (1 mg/day); (11) intact+DHT (1 mg/day); (12) intact+Vehicle. Dosing solutions were prepared daily by dissolving drug in dimethyl sulfoxide (DMSO) and diluting in polyethylene glycol 300 (PEG 300). All doses were administered for 120 days via daily subcutaneous injections in a volume of 0.20 ml.

Whole Body DEXA Analysis

Total body bone mineral density (BMD), percent fat mass (FM), and body weight (BW) were determined by dual energy x-ray absorptiometry (DEXA) (GE, Lunar Prodigy™) using the small animal software (Lunar enCORE, version 6.60.041) on days 0 and 120. Total body data was obtained by selecting an area encompassing the entire animal as the region of interest during data processing. For scanning, the animals were anesthetized with ketariine:xylazine (87:13 mg/kg).

Ex vivo DEXA Analysis

Immediately following the whole body DEXA scan on day 120, groups 1-11 were sacrificed, and the lumbar vertebrae, femurs, and tibia were excised and cleared of soft tissue. Group 12 was sacrificed at day 210. The excised bones were scanned through a 3 in deep room temperature water bath, to simulate soft tissue and prevent beam-hardening effects. The L2-L4 vertebrae were selected as a region of interest and analyzed for BMD by DEXA.

Femoral pQCT Analysis and Biomechanical Testing

The right femurs from groups 5, 6, 7, 9, 10, and 12 were sent to Skeletech, Inc. (Bothell, Wash.) for pQCT analysis and biomechanical testing. The femur was subject to pQCT scan using a Stratec XCT RM and associated software (Stratec Medizintechnik GmbH, Pforzheim, Germany. Software version 5.40 C). The femur was scanned at the mid-shaft. The position was verified using scout views and scan results from one 0.5 mm slice perpendicular to the long axis of the femur. Cortical bone mineral content was analyzed. After pQCT analysis, the de-fleshed whole femur was used in the three point bending test. The femur was placed on the lower supports of a three point bending fixture with anterior side facing downward in an Instron Mechanical Testing Machine (Instron 4465 retrofitted to 5500). The upper loading device was aligned to the center of the femoral shaft. The load was applied at a constant displacement rate of 6 mm/min until the femur broke. The location of maximal load was selected manually and values were calculated by machine's software (Merlin II, Instron). Maximum load was measured directly by the mechanical testing machine. The length between two supports was set to 14mm.

Statistics

The results were analyzed using Tukey's honestly significant difference method in a one-way analysis of variance and expressed as mean ±S.E.M. Significance was reported when P<0.05. SPSS 11.5.0 (Chicago, Ill.) was used to analyze the results.

Results

Whole Body DEXA Analysis

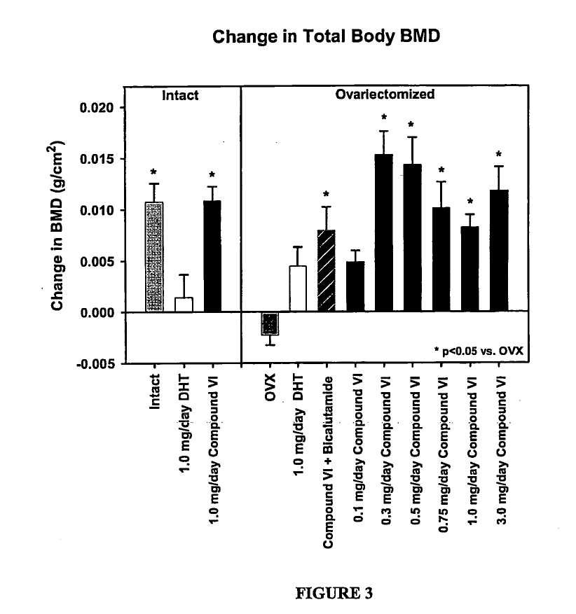

FIG. 3 shows the change in BMD between day 0 and day 120. As expected, OVX animals lost BMD during the course of the study. Compound VI treatment was able to prevent the loss of whole body BMD in OVX animals (i.e., no significant differences from intact control animals were observed) at doses greater than 0.1 mg/day. BMD changes following ovariectomy were not significantly different from OVX controls in the DHT or 0.1 mg/day Compound VI treated groups. Co-administration of bicalutarnide, a pure antiandrogen, was able to partially prevent the drug effects suggesting that the AR was important for regulating the bone response to Compound VI. The BMD change in intact and OVX groups receiving DHT was not different. In intact animals, DHT prevented the age related increase in BMD. In OVX animals, the effect of DHT treatment was not different from OVX controls.

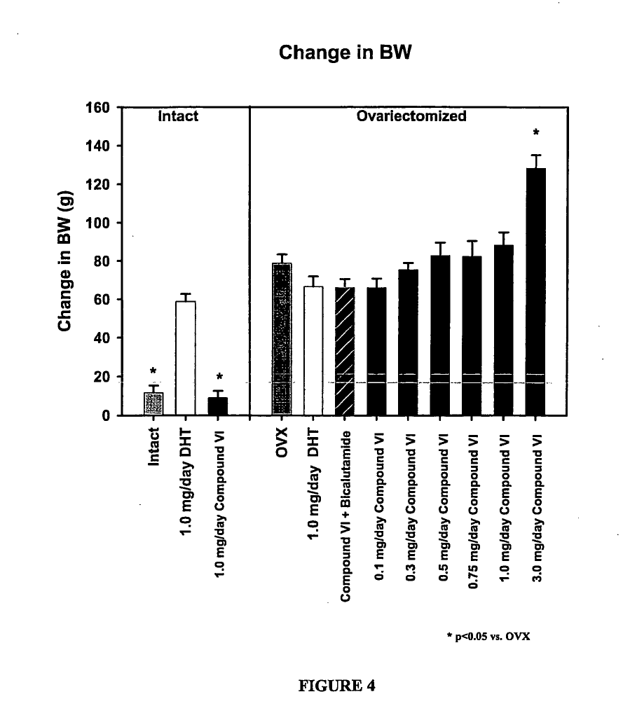

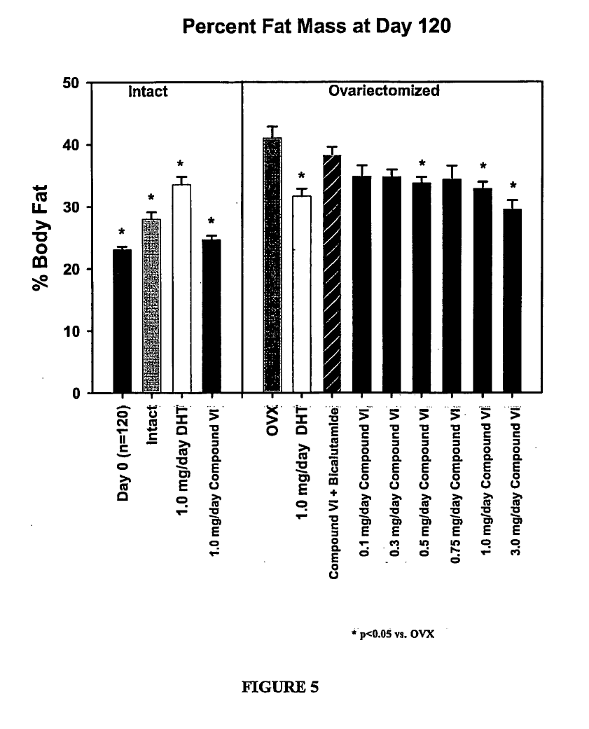

With the exception of the intact Compound VI and intact control groups, all groups gained a significant amount of body weight. In the Compound VI treated groups, we observed a dose-dependent trend in body weight gain, however the difference between drug treated groups and the OVX control group was negligible, except at the highest dose (FIG. 4). Even though the 3.0 mg/day dose group gained more weight than the other groups, their percent body fat was not different from intact controls. Fat Mass (FM) at day 120 is summarized in FIG. 5. A dose dependent decrease in percent fat mass of OVX animals was also observed. Additionally, the mean percent fat mass was lower in intact Compound VI treated animals than intact controls. None of the Compound VI treated groups were different from the intact group at day 120.

Excised Bone DEXA Analysis

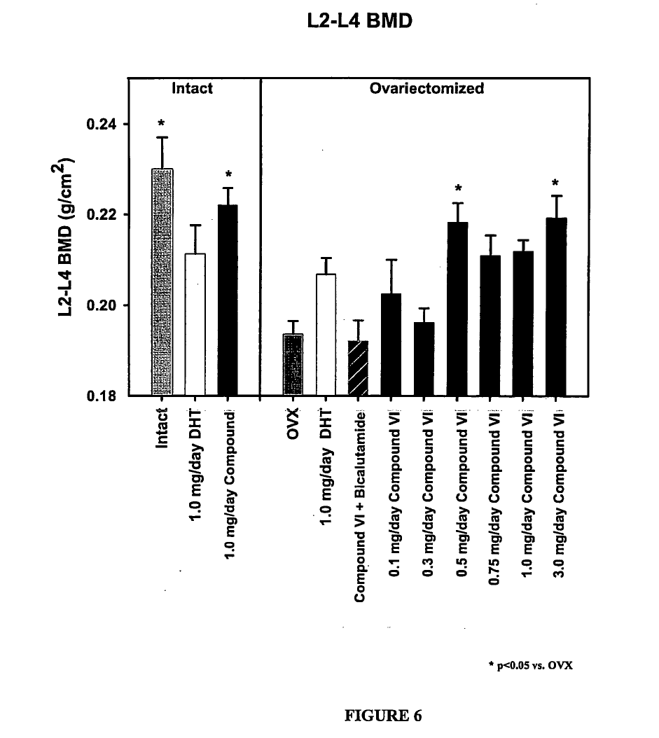

Vertebral fractures are the most common of all skeletal fractures (Melton, L. J., 3rd and S. R. Cummings, Heterogeneity of age-related fractures: implications for epidemiology. Bone Miner, 1987. 2(4): p. 321-31). The lumbar vertebrae contain a large volume of trabecular bone and demonstrate rapid bone loss following OVX or menopause. Therefore, the BMD in the L2-L4 vertebrae, a model to evaluate therapeutic effects on trabecular bone, was examined. As expected, OVX control animals showed significantly lower BMD in this region than intact controls. FIG. 6 demonstrates the bone-sparing effect of Compound VI in the L2-L4 vertebrae. A dose-dependent drug effect in the lumbar vertebrae was observed. L2-L4 BMD in groups receiving greater than 0.3 mg/day was not significantly different from intact controls and the 0.5 and 3.0 mg/kg dose groups exhibited a significantly higher BMD than the OVX control group. Co-administration of bicalutamide was able to completely block the effects of Compound VI, indicating the protective effects of SARMs are mediated through the AR. As seen in whole body BMD data, DHT treatment in intact animals caused a reduction in L2-L4 BMD, while in OVX animals DHT was only able to partially maintain the L2-L4 BMD.

Femoral PQCT Analysis and Biomechanical Testing

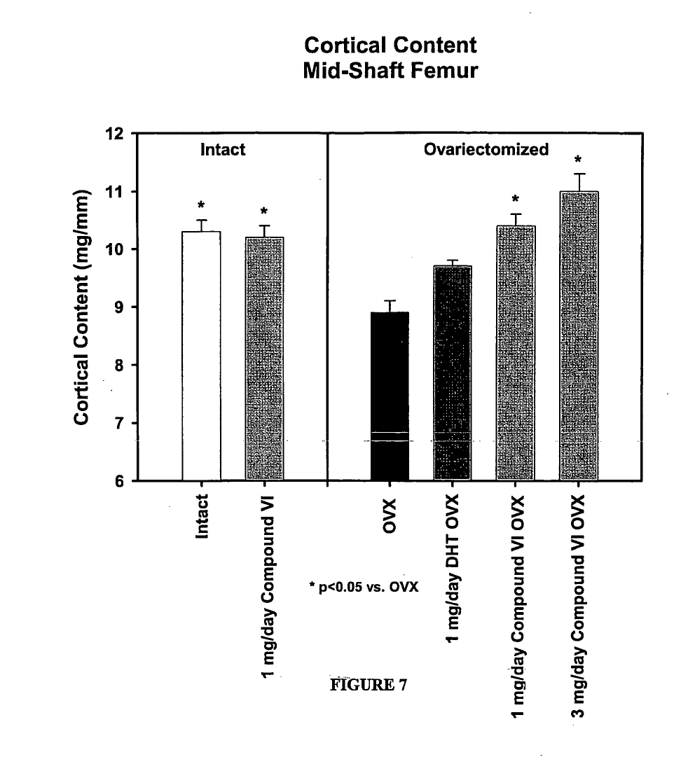

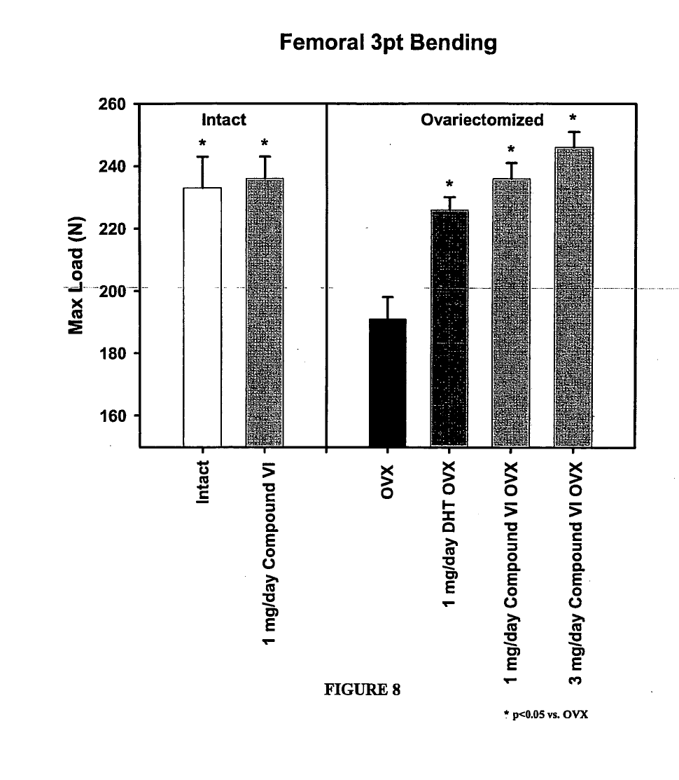

To determine the effect of Compound VI in cortical bone, the Cortical Content (CC-FIG. 7) and biomechanical strength (FIG. 8) of the mid-shaft of the femur was examined by pQCT and three-point bending. pQCT analysis of the femur showed that Compound VI was able to completely prevent the loss in CC observed following OVX at a dose of 1.0 mg/day. A 3.0 mg/day dose was able to increase the CC in OVX animals above that of intact animals. However, a larger sample size would be required to reach significance. Although it appears DHT treatment partially prevented the loss in CC, these results were not significant. CC in intact animals receiving Compound VI was not different from intact controls. A significant decrease in the biomechanical strength of the femur following OVX was observed. The effect of Compound VI on femur strength showed the same pattern of results as the effect observed in cortical content. The 1.0 mg/day dose was able to prevent the loss of bone strength, while the 3.0 mg/day dose resulted in an increase in maximum load. Although significant differences in cortical content were not observed between DHT and OVX controls, DHT was able to prevent the loss in bone strength.

The results show that OVX induced changes in whole body BMD, percent fat mass, body weight, L2-L4 BMD, femoral CC, and femoral biomechanical strength are modulated by a nonsteroidal SARM. Compound VI treatment resulted in dose-dependent increase in whole body BMD, body weight, L2-L4 BMD, femoral CC, and femoral biomechanical strength, while decreasing the percent fat mass in OVX animals. These studies have shown that Compound VI can be used as a treatment for osteoporosis.

It will be appreciated by a person skilled in the art that the present invention is not limited by what has been particularly shown and described hereinabove.

1. A method of treating a female subject suffering from an Androgen Deficiency in Female (ADIF)-associated condition, said method comprising the step of administering to said subject a selective androgen receptor modulator (SARM) compound, in an amount effective to treat said ADIF-associated condition.

2. The method of claim 1, wherein said method comprises administering an analog, derivative, isomer, metabolite, pharmaceutically acceptable salt, pharmaceutical product, hydrate, N-oxide, crystal, polymorph or prodrug of said SARM compound, or any combination thereof.

3. The method according to claim 1, wherein said SARM compound is represented by the structure of formula I: 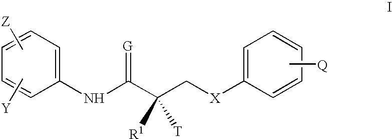
wherein G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alcyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 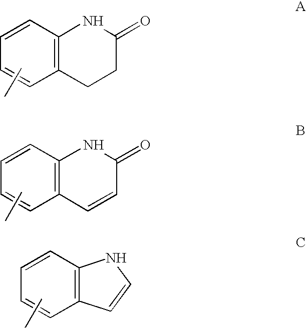
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

4. The method according to claim 1, wherein said SARM compound is represented by the structure of formula II. 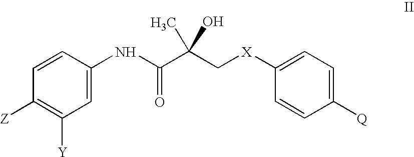
wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 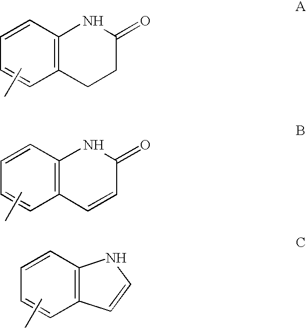
R is allyl, haloalkyl, dihaloalkyl, trihaloalkyl, CH₂F, CHF₂, CF₃, CF₂CF₃, aryl, phenyl, halogen, alkenyl or OH.

5. The method according to claim 1, wherein said SARM compound is represented by the structure of formula III. 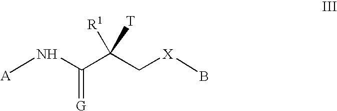
wherein X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 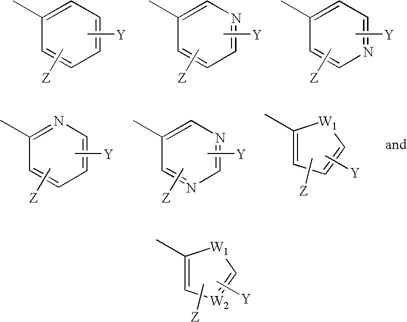 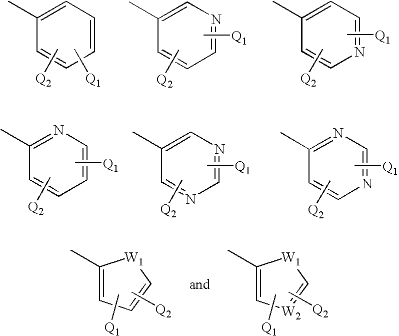
wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR CONHR NHCSCH₃, NHCSCF₃, NHCSR NHSO₂CH₃, NHSO₂R, OR, COR, OCOR, OSO₂R, SO₂R, SR, 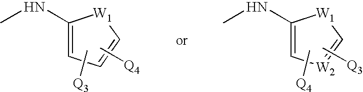
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is Nor NO.

6. The method according to claim 1, wherein said SARM compound is represented by the structure of formula IV: 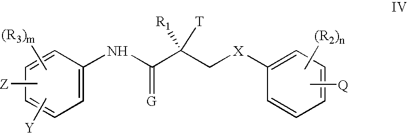
wherein X is a bond, O, CH₂, NH, Se, PR, NO or NR;
G is O or S,
T is OH, OR, —NHCOCH₃, or NHCOR;
R is alkyl, haloalkyl, dihaloalkyl, trihaloalkyl, CH₂F, CBF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 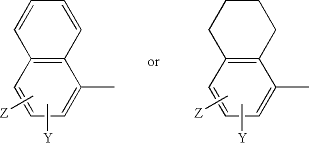
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 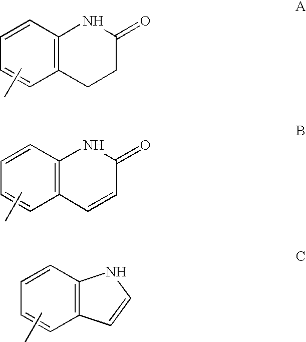
n is an integer of 1-4; and
m is an integer of 1-3.

7. The method according to claim 1, wherein said SARM compound is represented by the structure of formula V: 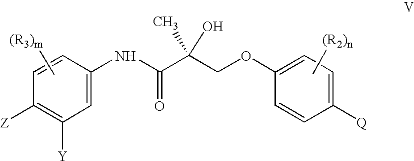
wherein
R₂ is F, Cl, Br, I, CH₃, CF₃, OH, CN, NO₂, NHCOCH₃, NHCOCF₃, NHCOR, alkyl, arylalkyl, OR, NH₂, NHR NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 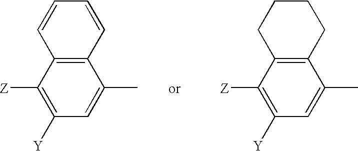
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 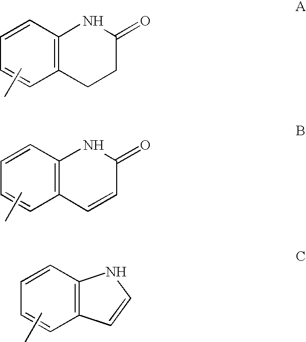
n is an integer of 1-4; and
m is an integer of 1-3.

8. The method according to claim 1, wherein said SARM compound is represented by the structure of formula VI.

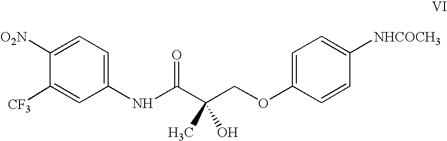

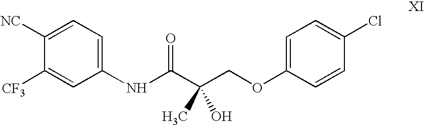

14. The method of claim 1, wherein the SARM is an androgen receptor agonist.

15. The method of claim 1, wherein the SARM has in-vivo androgenic and anabolic activity of a nonsteroidal ligand for the androgen receptor.

16. The method of claim 1, wherein the SARM is an androgen receptor antagonist.

17. The method of claim 1, wherein said SARM has an agonistic effect muscle or bone.

18. The method of claim 1, wherein said SARM has no effect on muscle or bone.

19. The method of claim 1, wherein said SARM penetrates the central nervous system (CNS).

20. The method of claim 1, wherein said SARM does not penetrate the central nervous system (CNS).

21. The method according to claim 1, wherein said administering comprises administering a pharmaceutical preparation comprising said SARM and/or its analog, derivative, isomer, metabolite, pharmaceutically acceptable salt, pharmaceutical product, hydrate, N-oxide, crystal, polymorph, prodrug, or any combination thereof; and a pharmaceutically acceptable carrier.

22. The method according to claim 21, wherein said administering comprises intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

23. The method according to claim 21 wherein said pharmaceutical preparation is a pellet, a tablet, a capsule, a solution, a suspension, an emulsion, an elixir, a gel, a cream, a suppository or a parenteral formulation.

24. The method of claim 1, wherein said ADIF-associated condition is sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer, ovarian cancer, or any combination thereof.

25. The method of claim 1, wherein said female subject is an aging female subject.

26. A method of preventing, suppressing, inhibiting or reducing the incidence of an Androgen Deficiency in Female (ADIF)-associated condition in a female subject, said method comprising the step of ad-ministering to said subject a selective androgen receptor modulator (SARM) compound, in an amount effective to prevent, suppress, inhibit or reduce the incidence of said ADIF-condition.

27. The method of claim 26, wherein said method comprises administering an analog, derivative, isomer, metabolite, pharmaceutically acceptable salt, pharmaceutical product, hydrate, N-oxide, crystal, polymorph or prodrug of said SARM compound, or any combination thereof.

28. The method according to claim 26, wherein said SARM compound is represented by the structure of formula I: 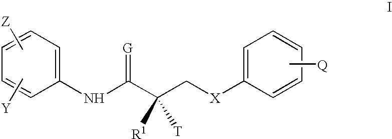
wherein G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is allyl, halogen, CF₃, CN CR₃, SnR₃, NR₂, NHCOCH₃, NHCOCF₃, NHCOR, NHCONHR, NHCOOR, OCONHR, CONR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 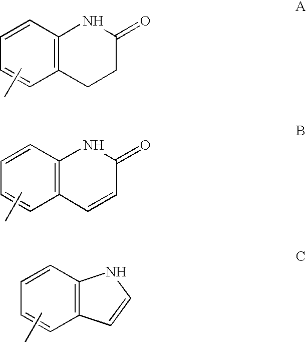
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

29. The method according to claim 26, wherein said SARM compound is represented by the structure of formula II. 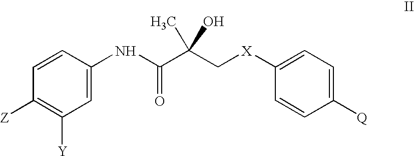
wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is allyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO2R, OR, COR, OCOR, OSO₂R, SO₂R, SR; or Q together with the benzene ring to which it is attached is a fused ring system represented by structure A, B or C: 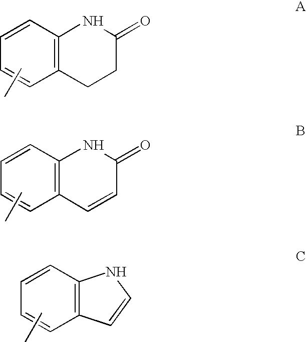
R is alkyl, haloalkyl, dihaloalkyl, trihaloalkyl, CH₂F, CHF₂, CF₃, CF₂CF₃, aryl, phenyl, halogen, alkenyl or OH.

30. The method according to claim 26, wherein said SARM compound is represented by the structure of formula III. 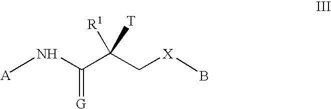
wherein X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 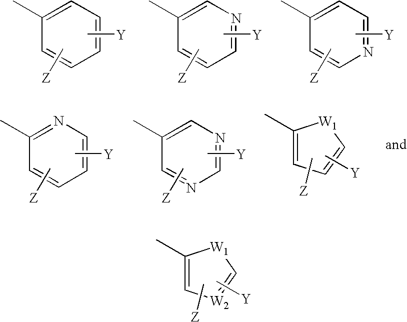
B is a ring selected from: 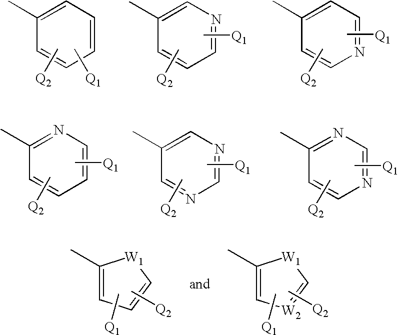
wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 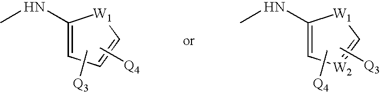
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

31. The method according to claim 26, wherein said SARM compound is represented by the structure of formula IV: 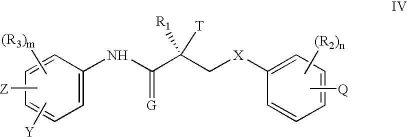
wherein X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 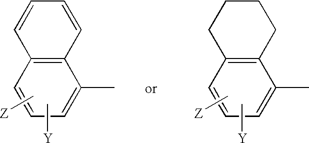
Z is NO₂, CN, COR, COOH, or CONHR,
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 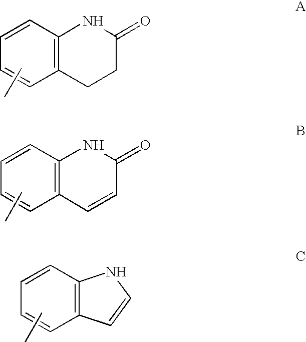
n is an integer of 1-4; and
m is an integer of 1-3.

32. The method according to claim 26, wherein said SARM compound is represented by the structure of formula V: 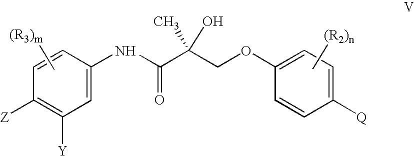
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 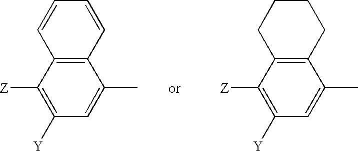
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 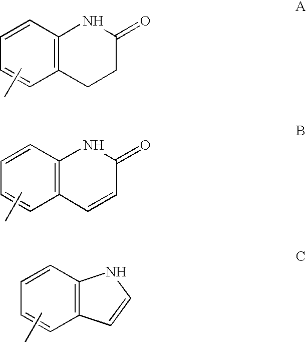
n is an integer of 1-4; and
m is an integer of 1-3.

33. The method according to claim 26, wherein said SARM compound is represented by the structure of formula VI.

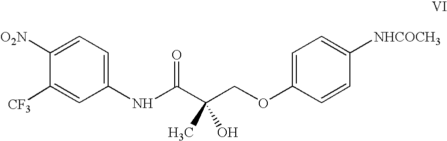

34. The method according to claim 26, wherein said SARM compound is represented by the structure of formula VII.

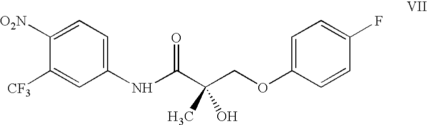

35. The method according to claim 26, wherein said SARM compound is represented by the structure of formula VIII.

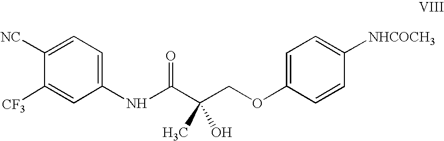

36. The method according to claim 26, wherein said SARM compound is represented by the structure of formula IX.

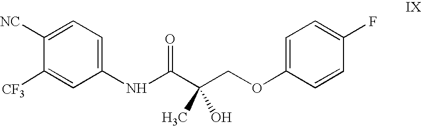

37. The method according to claim 26, wherein said SARM compound is represented by the structure of formula X.

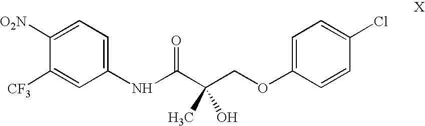

38. The method according to claim 26, wherein said SARM compound is represented by the structure of formula XI.

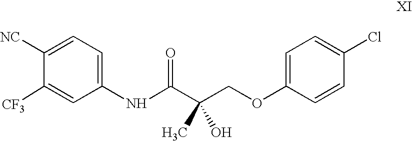

39. The method of claim 26, wherein the SARM is an androgen receptor agonist.

40. The method of claim 26, wherein the SARM has in-vivo androgenic and anabolic activity of a nonsteroidal ligand for the androgen receptor.

41. The method of claim 26, wherein the SARM is an androgen receptor antagonist.

42. The method of claim 26, wherein said SARM has an agonistic effect muscle or bone.

43. The method of claim 26, wherein said SARM has no effect on muscle or bone.

44. The method of claim 26, wherein said SARM penetrates the central nervous system (CNS).

45. The method of claim 26, wherein said SARM does not penetrate the central nervous system (CNS).

46. The method according to claim 26, wherein said administering comprises administering a pharmaceutical preparation comprising said SARM and/or its analog, derivative, isomer, metabolite, pharmaceutically acceptable salt, pharmaceutical product, hydrate, N-oxide, crystal, polymorph, prodrug, or any combination thereof; and a pharmaceutically acceptable carrier.

47. The method according to claim 46, wherein said administering comprises intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

48. The method according to claim 46 wherein said pharmaceutical preparation is a pellet, a tablet, a capsule, a solution, a suspension, an emulsion, an elixir, a gel, a cream, a suppository or a parenteral formulation.

49. The method of claim 26, wherein said ADIF-associated condition is sexual dysfimction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer, ovarian cancer, or any combination thereof.

50. The method of claim 26, wherein said female subject is an aging female subject.

51. A method of treating a female subject suffering from sexual dysfimction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer or ovarian cancer due to Androgen Deficiency in Female (ADIF), said method comprising the step of administering to said subject a selective androgen receptor modulator (SARM) compound.

52. The method of claim 51, wherein said method comprises administering an analog, derivative, isomer, metabolite, pharmaceutically acceptable salt, pharmaceutical product, hydrate, N-oxide, crystal, polymorph or prodrug of said SARM compound, or any combination thereof.

53. The method according to claim 51, wherein said SARM compound is represented by the structure of formula I: 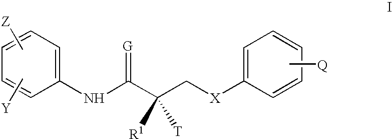
wherein G is O or S;
X is a bond, O, CH₂, NH, Se, PR, NO or NR;
T is OH, OR, —NHCOCH₃, or NHCOR
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, or Q together with the benzene ring to which it is attached is a fused ring system represented by structure A, B or C: 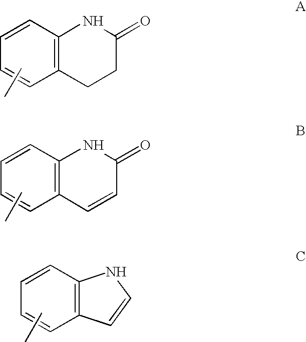
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

54. The method according to claim 51, wherein said SARM compound is represented by the structure of formula II. 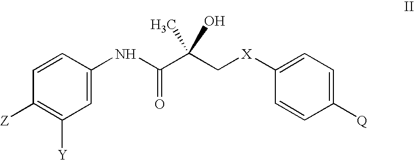
wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 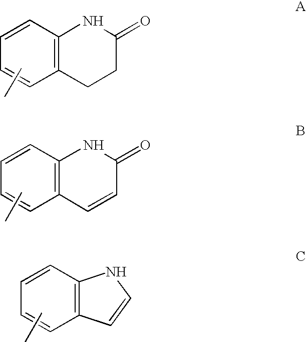
R is alkyl, haloalkyl, dihaloalkyl, trihaloalkyl, CH₂F, CHF₂, CF₃, CF₂CF₃, aryl, phenyl, halogen, alkenyl or OH.

55. The method according to claim 51, wherein said SARM compound is represented by the structure of formula III. 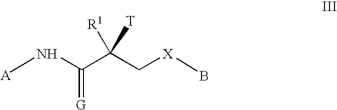
wherein X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 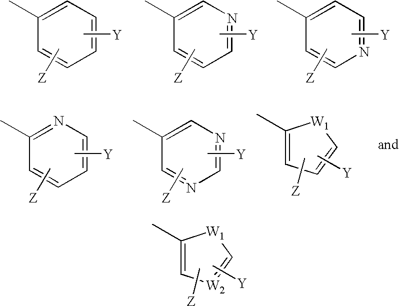
B is a ring selected from: 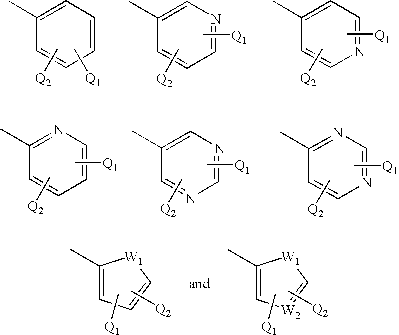
wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 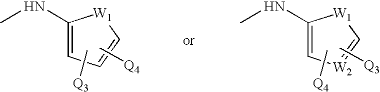
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

56. The method according to claim 51, wherein said SARM compound is represented by the structure of formula IV: 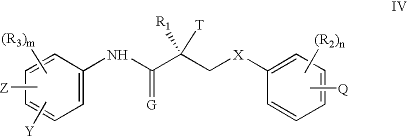
wherein X is a bond, O, CH₂, NH, Se, PR, NO or NR;
G is O or S;
T is OH, OR, —NHCOCH₃, or NHCOR;,
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, all, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 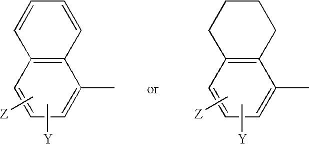
Z is NO₂, CN, COR, COOH, or CONHR;
Y is CF₃, F, Br, Cl, I, CN, or SnR₃;
Q is H, alkyl halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 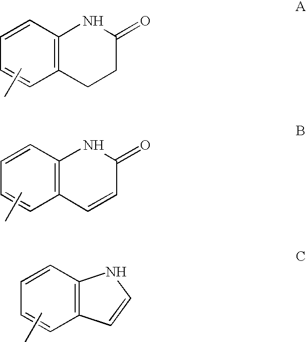
n is an integer of 1-4; and
m is an integer of 1-3.

57. The method according to claim 51, wherein said SARM compound is represented by the structure of formula V: 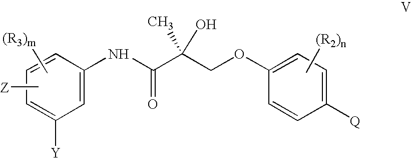
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 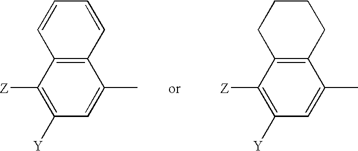
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃ F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 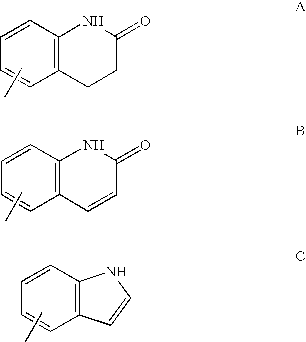
n is an integer of 1-4; and
m is an integer of 1-3.

58. The method according to claim 51, wherein said SARM compound is represented by the structure of formula VI.

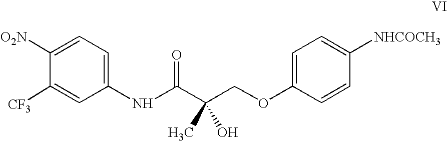

59. The method according to claim 51, wherein said SARM compound is represented by the structure of formula VII.

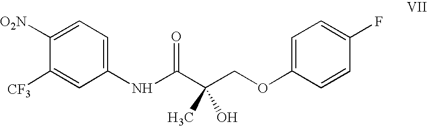

60. The method according to claim 51, wherein said SARM compound is represented by the structure of formula VIII.

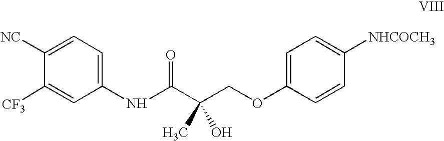

61. The method according to claim 51, wherein said SARM compound is represented by the structure of formula IX.

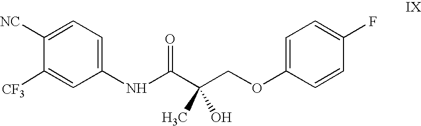

62. The method according to claim 51, wherein said SARM compound is represented by the structure of formula X.

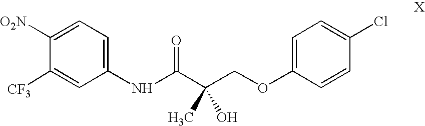

63. The method according to claim 51, wherein said SARM compound is represented by the structure of formula XI.

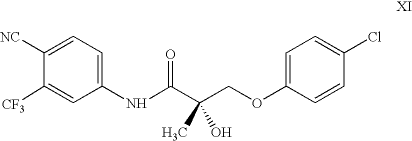

64. The method of claim 51, wherein the SARM is an androgen receptor agonist.

65. The method of claim 51, wherein the SARM has in-vivo androgenic and anabolic activity of a nonsteroidal ligand for the androgen receptor.

66. The method of claim 51, wherein the SARM is an androgen receptor antagonist.

67. The method of claim 51, wherein said SARM has an agonistic effect muscle or bone.

68. The method of claim 51, wherein said SARM has no effect on muscle or bone.

69. The method of claim 51, wherein said SARM penetrates the central nervous system (CNS).

70. The method of claim 51, wherein said SARM does not penetrate the central nervous system (CNS).

71. The method according to claim 51, wherein said administering comprises administering a pharmaceutical preparation comprising said SARM and/or its analog, derivative, isomer, metabolite, pharmaceutically acceptable salt, pharmaceutical product, hydrate, N-oxide, crystal, polymorph, prodrug, or any combination thereof; and a pharmaceutically acceptable carrier.

72. The method according to claim 71, wherein said administering comprises intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

73. The method according to claim 71 wherein said pharmaceutical preparation is a pellet, a tablet, a capsule, a solution, a suspension, an emulsion, an elixir, a gel, a cream, a suppository or a parenteral formulation.

74. The method of claim 51, wherein said ADIF-associated condition is sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer, ovarian cancer, or any combination thereof.

75. The method of claim 51, wherein said female subject is an aging female subject.

76. A method of preventing, suppressing, inhibiting or reducing the incidence of an Androgen Deficiency in Female (ADIF)-associated condition selected from sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer and ovarian cancer, in a female subject, said method comprising the step of administering to said subject a selective androgen receptor modulator (SARM) compound.

77. The method of claim 76, wherein said method comprises administering an analog, derivative, isomer, metabolite, pharmaceutically acceptable salt, pharmaceutical product, hydrate, N-oxide, crystal, polymorph or prodrug of said SARM compound, or any combination thereof.

78. The method according to claim 76, wherein said SARM compound is represented by the structure of formula I: 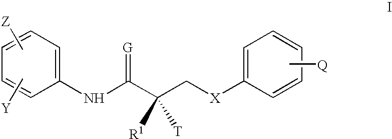
wherein G is O or S;
X is a bond, O, CH₂, NH, Se, PR, NO or NR;
T is OH, OR, —NHCQCH₃, or NHCOR
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 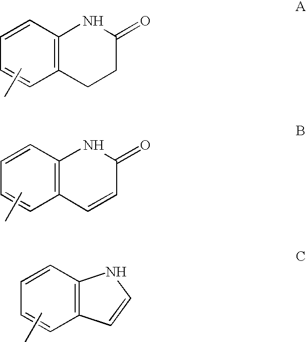
R is alkyl, haloalkyl, dihaloalkyl, trihaloalkyl, CH₂F, CHF₂, CF₃, CF₂CF₃, aryl, phenyl, halogen, alkenyl or OH; and
R₁ is CH₃, CH₂F, CHF₂, CF₃, CH₂CH₃, or CF₂CF₃.

79. The method according to claim 76, wherein said SARM compound is represented by the structure of formula II. 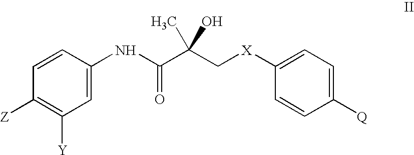
wherein X is a bond, O, CH₂, NH, Se, PR, NO or NR;
Z is NO₂, CN, COOH, COR, NHCOR or CONHR;
Y is CF₃, F, I, Br, Cl, CN, CR₃ or SnR₃;
Q is alkyl, halogen, CF₃, CN CR₃, SnR₃, NR₂, NHCOCH₃, NHCOCF₃, NHCOR, NHCONHR, NHCOOR, CONHR, CONHR, NHCSCH₃, NHCSCF₃, NHCSR NHSO₂CH₃, NHSO₂R, OR, COR, OCOR, OSO₂R, SO₂R, SR; or Q together with the benzene ring to which it is attached is a fused ring system represented by structure A, B or C: 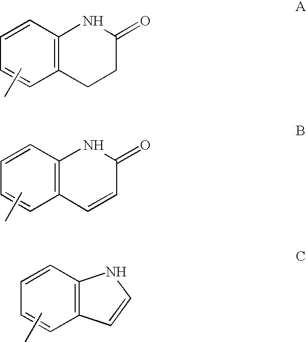
R is alkyl, haloalkyl, dihaloalkyl, trihaloalkyl, CH₂F, CHF₂, CF₃, CF₂CF₃, aryl, phenyl, halogen, alkenyl or OH.

80. The method according to claim 76, wherein said SARM compound is represented by the structure of formula III. 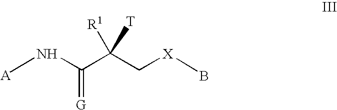
wherein X is a bond, O, CH₂, NH, Se, PR, NO or NR;
G is O or S;
R₁ is CH₃, CH₂F, CHF₂, CF₃, CH₂CH₃, or CF₂CF₃;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
A is a ring selected from: 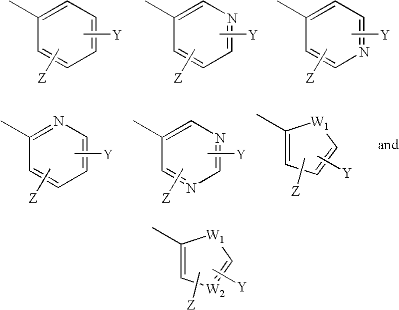
B is a ring selected from: 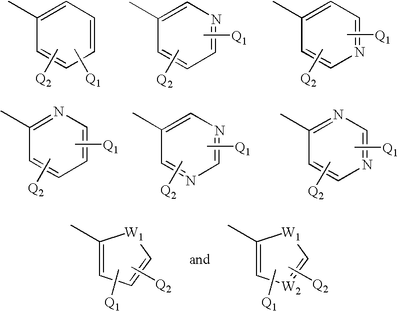
wherein A and B cannot simultaneously be a benzene ring;
Z is NO₂, CN, COOH, COR, NHCOR or CONHR;
Y is CF₃, F, I, Br, Cl, CN CR₃ or SnR₃;
Q₁ and Q₂ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R, SR, 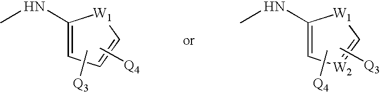
Q₃ and Q₄ are independently of each other a hydrogen, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OR, COR, OCOR, OSO₂R, SO₂R or SR;
W₁ is O, NH, NR, NO or S; and
W₂ is N or NO.

81. The method according to claim 76, wherein said SARM compound is represented by the structure of formula IV: 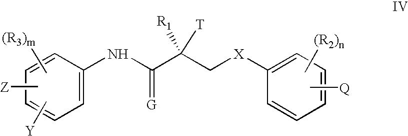
wherein X is a bond, O, CH₂, NH, Se, PR, NO or NR;
G is O or S;
T is OH, OR, —NHCOCH₃, or NHCOR;
R is alkyl, haloalkyl, dihaloalkyl, trihaloalkyl, CH₂F, CHF₂, CF₃, CF₂CF₃, aryl, phenyl, halogen, alkenyl or OH;
R₁ is CH₃, CH₂F, CHF₂, CF₃, CH₂CH₃, or CF₂CF₃;
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 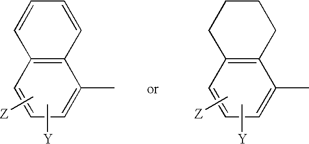
Z is NO₂, CN, COR, COOH, or CONHR;
Y is CF₃ F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 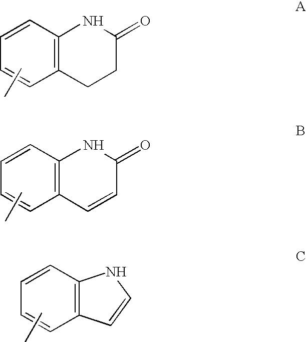
n is an integer of 1-4; and
m is an integer of 1-3.

82. The method according to claim 76, wherein said SARM compound is represented by the structure of formula V: 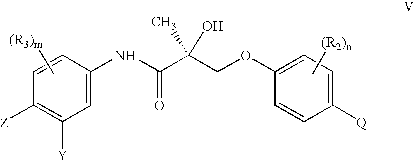
wherein
R₂ is F, Cl, Br, I, CH₃, CF₃, OH, CN, NO₂, NHCOCH₃, NHCOCF₃, NHCOR, alkyl, arylalkyl, OR, NH₂, NHR, NR₂, SR;
R₃ is F, Cl, Br, I, CN, NO₂, COR, COOH, CONHR, CF₃, SnR₃, or R₃ together with the benzene ring to which it is attached forms a fused ring system represented by the structure: 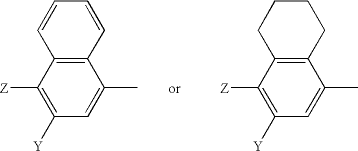
R is alkyl, haloalkyl, dihaloalkyl, trihaloalkyl, CH₂F, CHF₂, CF₃, CF₂CF₃, aryl, phenyl, halogen, alkenyl or OH;
Z is NO₂, CN, COR, COOH, or CONHR;
Y is CF₃, F, Br, Cl, I, CN, or SnR₃;
Q is H, alkyl, halogen, CF₃, CN CR₃, SnR₃, NR₂, NHCOCH₃, NHCOCF₃, NHCOR, NHCONHR, NHCOOR, OCONHR, CONHR, NHCSCH₃, NHCSCF₃, NHCSR NHSO₂CH₃, NHSO₂R, OH, OR, COR, OCOR, OSO₂R, SO₂R, SR; or Q together with the benzene ring to which it is attached is a fused ring system represented by structure A, B or C: 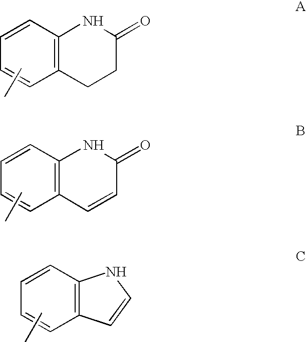
n is an integer of 1-4; and
m is an integer of 1-3.

83. The method according to claim 76, wherein said SARM compound is represented by the structure of formula VI.

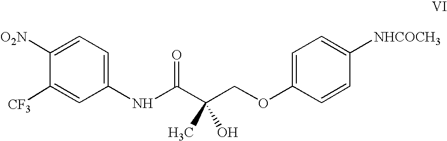

84. The method according to claim 76, wherein said SARM compound is represented by the structure of formula VII.

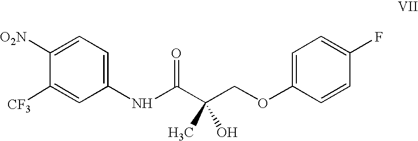

85. The method according to claim 76, wherein said SARM compound is represented by the structure of formula VIII.

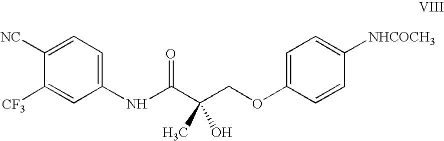

86. The method according to claim 76, wherein said SARM compound is represented by the structure of formula IX.

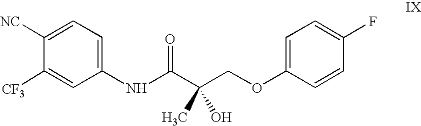

87. The method according to claim 76, wherein said SARM compound is represented by the structure of formula X.

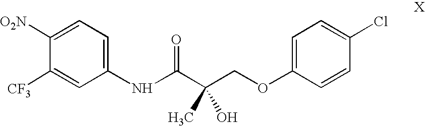

88. The method according to claim 76, wherein said SARM compound is represented by the structure of formula XI.

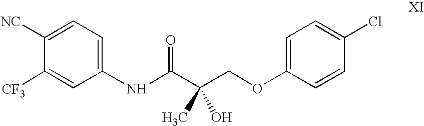

89. The method of claim 76, wherein the SARM is an androgen receptor agonist.

90. The method of claim 76, wherein the SARM has in-vivo androgenic and anabolic activity of a nonsteroidal ligand for the androgen receptor.

91. The method of claim 76, wherein the SARM is an androgen receptor antagonist.

92. The method of claim 76, wherein said SARM has an agonistic effect muscle or bone.

93. The method of claim 76, wherein said SARM has no effect on muscle or bone.

94. The method of claim 76, wherein said SARM penetrates the central nervous system (CNS).

95. The method of claim 70, wherein said SARM does not penetrate the central nervous system (CNS).

96. The method according to claim 76, wherein said administering comprises administering a pharmaceutical preparation comprising said SARM and/or its analog, derivative, isomer, metabolite, pharmaceutically acceptable salt, pharmaceutical product, hydrate, N-oxide, crystal, polymorph, prodrug, or any combination thereof; and a pharmaceutically acceptable carrier.

97. The method according to claim 96, wherein said administering comprises intravenously, intraarterially, or intramuscularly injecting to said subject said pharmaceutical preparation in liquid form; subcutaneously implanting in said subject a pellet containing said pharmaceutical preparation; orally administering to said subject said pharmaceutical preparation in a liquid or solid form; or topically applying to the skin surface of said subject said pharmaceutical preparation.

98. The method according to claim 96 wherein said pharmaceutical preparation is a pellet, a tablet, a capsule, a solution, a suspension, an emulsion, an elixir, a gel, a cream, a suppository or a parenteral formulation.

99. The method of claim 76, wherein said ADIF-associated condition is sexual dysfunction, decreased sexual libido, hypogonadism, sarcopenia, osteopenia, osteoporosis, alterations in cognition and mood, fatigue, depression, anemia, muscle weakness, hair loss, obesity, polycystic ovarian disease, endometriosis, breast cancer, uterine cancer, ovarian cancer, or any combination thereof.

100. The method of claim 76, wherein said female subject is an aging female subject.
